(19) United States
(12) Patent Application Publication
(10) Pub. No.: US 2023/0201374 A1
(43) Pub. Date: Jun. 29, 2023

(54) GENE THERAPY AND TARGETED DELIVERY OF CONJUGATED COMPOUNDS

(71) Applicant: Ionis Pharmaceuticals, Inc., Carlsbad, CA (US)

(72) Inventor: Frank Rigo, Carlsbad, CA (US)

(73) Assignee: Ionis Pharmaceuticals, Inc., Carlsbad, CA (US)

(21) Appl. No.: 17/812,401

(22) Filed: Jul. 13, 2022

Related U.S. Application Data
(63) Continuation of application No. 17/544,839, filed on Dec. 7, 2021, now abandoned.
Application No. 17/544,839 is a continuation of application No. 16/335,171, filed on Mar. 20, 2019, now abandoned.
(60) Provisional application No. 62/399,233, filed on Sep. 23, 2016.

(51) Int. Cl. A61K 48/00; A61K 47/54; A61K 9/00; A61K 31/7115; A61K 31/712; A61K 31/7125; A61K 35/30; C07K 14/705

(52) U.S. Cl. CPC A61K 48/0066; A61K 47/549; A61K 9/0019; A61K 31/7115; A61K 31/712; A61K 31/7125; A61K 35/30; A61K 48/0016; A61K 48/0075; C07K 14/705

(57) ABSTRACT

Provided herein are methods, compounds, and compositions useful for targeted delivery of compounds to non-native cells ectopically expressing cell surface receptors. Such methods, compounds, and compositions are useful, for example, in gene therapy mediated ectopic expression of cell surface receptors and targeted delivery of compounds, such as conjugated oligonucleotides, to the non-native cells ectopically expressing cell surface receptors. Such methods, compounds, and compositions can be useful, for example, to treat, prevent, delay or ameliorate disease in an individual by targeted reduction of a gene of interest in the non-native cell ectopically expressing cell surface receptors.

### SEQUENCE LISTING

The present application is being filed along with a Sequence Listing in electronic format. The Sequence Listing is provided as a file entitled BIOL0311USC2SEQ_ST26.xml, created Jul. 12, 2022 which is 3 Kb in size. The information in the electronic format of the sequence listing is incorporated herein by reference in its entirety.

### FIELD

Provided herein are methods, compounds, and compositions useful for targeted delivery of compounds to non-native cells ectopically expressing cell surface receptors. Such methods, compounds, and compositions are useful, for example, in gene therapy mediated ectopic expression of cell surface receptors and targeted delivery of compounds, such as conjugated oligonucleotides, to the non-native cells ectopically expressing cell surface receptors. Such methods, compounds, and compositions can be useful, for example, to treat, prevent, delay or ameliorate disease in an individual by targeted reduction of a gene of interest in the non-native cell ectopically expressing cell surface receptors.

### BACKGROUND

Recent gene therapy clinical trials showing safety and efficacy have rekindled the field's interest in gene therapy as a therapeutic platform. These positive clinical results reflect recent improvements in gene therapy vector design.

### SUMMARY

Certain embodiments are directed to use of gene therapy vectors to ectopically express a cell surface receptor in a cell that endogenously expresses no or low levels of the receptor. In certain embodiments, such a cell can be considered non-native in that it normally does not endogenously express the receptor or only expresses the receptor at low levels relative to a native cell that normally does endogenously express the receptor or expresses the receptor at high levels.

In certain embodiments, a non-native cell engineered to ectopically express a cell surface receptor by gene therapy can be targeted by a compound comprising a conjugate group that binds to the receptor. In certain embodiments, the conjugate group comprises a ligand that binds to the receptor.

### DETAILED DESCRIPTION

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

The section headings used herein are for organizational purposes only and are not to be construed as limiting the subject matter described. All documents, or portions of documents, cited in this application, including, but not limited to, patents, patent applications, articles, books, treatises, and GenBank and NCBI reference sequence records are hereby expressly incorporated by reference for the portions of the document discussed herein, as well as in their entirety.

It is understood that the sequence set forth in each SEQ ID NO in the examples contained herein is independent of any modification to a sugar moiety, an internucleoside linkage, or a nucleobase. As such, compounds defined by a SEQ ID NO may comprise, independently, one or more modifications to a sugar moiety, an internucleoside linkage, or a nucleobase. Compounds described by ISIS/IONIS number (ISIS/ION #) indicate a combination of nucleobase sequence, chemical modification, and motif.

Unless otherwise indicated, the following terms have the following meanings:

“2′-deoxynucleoside” means a nucleoside comprising 2′-H(H) furanosyl sugar moiety, as found in naturally occurring deoxyribonucleic acids (DNA). In certain embodiments, a 2′-deoxynucleoside may comprise a modified nucleobase or may comprise an RNA nucleobase (uracil).

“2′-O-methoxyethyl” (also 2′-MOE and 2′-O(CH₂)₂—OCH₃) refers to an O-methoxy-ethyl modification at the 2′ position of a furanosyl ring. A 2′-O-methoxyethyl modified sugar is a modified sugar.

“2′-MOE nucleoside” (also 2′-O-methoxyethyl nucleoside) means a nucleoside comprising a 2′-MOE modified sugar moiety.

“2′-substituted nucleoside” or “2-modified nucleoside” means a nucleoside comprising a 2′-substituted or 2′-modified sugar moiety. As used herein, “2′-substituted” or “2-modified” in reference to a sugar moiety means a sugar moiety comprising at least one 2′-substituent group other than H or OH.

“3′ target site” refers to the nucleotide of a target nucleic acid which is complementary to the 3′-most nucleotide of a particular compound.

“5′ target site” refers to the nucleotide of a target nucleic acid which is complementary to the 5′-most nucleotide of a particular compound.

“5-methylcytosine” means a cytosine with a methyl group attached to the 5 position.

“About” means within ±10% of a value. For example, if it is stated, “the compounds affected about 70% inhibition of a target”, it is implied that target levels are inhibited within a range of 60% and 80%.

“Administration” or “administering” refers to routes of introducing a compound or composition provided herein to an individual to perform its intended function. An example of a route of administration that can be used includes, but is not limited to parenteral administration, such as subcutaneous, intravenous, or intramuscular injection or infusion.

“Administered concomitantly” or “co-administration” means administration of two or more compounds in any manner in which the pharmacological effects of both are manifest in the patient. Concomitant administration does not require that both compounds be administered in a single pharmaceutical composition, in the same dosage form, by the same route of administration, or at the same time. The effects of both compounds need not manifest themselves at the same time. The effects need only be overlapping for a period of time and need not be coextensive. Concomitant administration or co-administration encompasses administration in parallel or sequentially.

“Amelioration” refers to an improvement or lessening of at least one indicator, sign, or symptom of an associated disease, disorder, or condition. In certain embodiments, amelioration includes a delay or slowing in the progression or severity of one or more indicators of a condition or disease. The progression or severity of indicators may be determined by subjective or objective measures, which are known to those skilled in the art.

“Animal” refers to a human or non-human animal, including, but not limited to, mice, rats, rabbits, dogs, cats, pigs, and non-human primates, including, but not limited to, monkeys and chimpanzees.

“Antisense activity” means any detectable and/or measurable activity attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound to the target.

“Antisense compound” means a compound comprising an oligonucleotide and optionally one or more additional features, such as a conjugate group or terminal group. Examples of antisense compounds include single-stranded and double-stranded compounds, such as, oligonucleotides, ribozymes, siRNAs, shRNAs, ssRNAs, and occupancy-based compounds.

“Antisense inhibition” means reduction of target nucleic acid levels in the presence of an antisense compound complementary to a target nucleic acid compared to target nucleic acid levels in the absence of the antisense compound.

“Antisense mechanisms” are all those mechanisms involving hybridization of a compound with target nucleic acid, wherein the outcome or effect of the hybridization is either target degradation or target occupancy with concomitant stalling of the cellular machinery involving, for example, transcription or splicing.

“Antisense oligonucleotide” means an oligonucleotide having a nucleobase sequence that is complementary to a target nucleic acid or region or segment thereof. In certain embodiments, an antisense oligonucleotide is specifically hybridizable to a target nucleic acid or region or segment thereof.

“Bicyclic nucleoside” or “BNA” means a nucleoside comprising a bicyclic sugar moiety. “Bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

“Branching group” means a group of atoms having at least 3 positions that are capable of forming covalent linkages to at least 3 groups. In certain embodiments, a branching group provides a plurality of reactive sites for connecting tethered ligands to an oligonucleotide via a conjugate linker and/or a cleavable moiety.

“Cell surface receptor” means any receptor that has at least a portion of the receptor on the surface of the cell. Cell-surface receptors include transmembrane receptors and extracellular domain receptors.

“Cell surface receptor upregulating agent” means any agent capable of increasing expression of a cell surface receptor.

“Cell-targeting moiety” means a conjugate group or portion of a conjugate group that is capable of binding to a particular cell type or particular cell types.

“cEt” or “constrained ethyl” means a bicyclic furanosyl sugar moiety comprising a bridge connecting the 4′-carbon and the 2′-carbon, wherein the bridge has the formula: 4′-CH(CH₃)—O-2′.

“Chemical modification” in a compound describes the substitutions or changes through chemical reaction, of any of the units in the compound. “Modified nucleoside” means a nucleoside having, independently, a modified sugar moiety and/or modified nucleobase. “Modified oligonucleotide” means an oligonucleotide comprising at least one modified internucleoside linkage, a modified sugar, and/or a modified nucleobase.

“Chemically distinct region” refers to a region of a compound that is in some way chemically different than another region of the same compound. For example, a region having 2′-O-methoxyethyl nucleotides is chemically distinct from a region having nucleotides without 2′-O-methoxyethyl modifications.

“Chimeric antisense compounds” means antisense compounds that have at least 2 chemically distinct regions, each position having a plurality of subunits.

“Cleavable bond” means any chemical bond capable of being split. In certain embodiments, a cleavable bond is selected from among: an amide, a polyamide, an ester, an ether, one or both esters of a phosphodiester, a phosphate ester, a carbamate, a di-sulfide, or a peptide.

“Cleavable moiety” means a bond or group of atoms that is cleaved under physiological conditions, for example, inside a cell, an animal, or a human.

“Complementary” in reference to an oligonucleotide means the nucleobase sequence of such oligonucleotide or one or more regions thereof matches the nucleobase sequence of another oligonucleotide or nucleic acid or one or more regions thereof when the two nucleobase sequences are aligned in opposing directions. Nucleobase matches or complementary nucleobases, as described herein, are limited to the following pairs: adenine (A) and thymine (T), adenine (A) and uracil (U), cytosine (C) and guanine (G), and 5-methyl cytosine (^{m}C) and guanine (G) unless otherwise specified. Complementary oligonucleotides and/or nucleic acids need not have nucleobase complementarity at each nucleoside and may include one or more nucleobase mismatches. By contrast, “fully complementary” or “100% complementary” in reference to oligonucleotides means that such oligonucleotides have nucleobase matches at each nucleoside without any nucleobase mismatches.

“Conjugate group” means a group of atoms that is attached to an oligonucleotide. Conjugate groups include a conjugate moiety and a conjugate linker that attaches the conjugate moiety to the oligonucleotide.

“Conjugate linker” means a group of atoms comprising at least one bond that connects a conjugate moiety to an oligonucleotide.

“Conjugate moiety” means a group of atoms that is attached to an oligonucleotide via a conjugate linker.

“Contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

“Designing” or “Designed to” refer to the process of designing a compound that specifically hybridizes with a selected nucleic acid molecule.

“Diluent” means an ingredient in a composition that lacks pharmacological activity, but is pharmaceutically necessary or desirable. For example, the diluent in an injected composition can be a liquid, e.g. saline solution.

“Differently modified” mean chemical modifications or chemical substituents that are different from one another, including absence of modifications. Thus, for example, a MOE nucleoside and an unmodified DNA nucleoside are “differently modified,” even though the DNA nucleoside is unmodified. Likewise, DNA and RNA are “differently modified,” even though both are naturally-occurring unmodified nucleosides. Nucleosides that are the same but for comprising different nucleobases are not differently modified. For example, a nucleoside comprising a 2′-OMe modified sugar and an unmodified adenine nucleobase and a nucleoside comprising a 2′-OMe modified sugar and an unmodified thymine nucleobase are not differently modified.

“Dose” means a specified quantity of a compound or pharmaceutical agent provided in a single administration, or in a specified time period. In certain embodiments, a dose may be administered in two or more boluses, tablets, or injections. For example, in certain embodiments, where subcutaneous administration is desired, the desired dose may require a volume not easily accommodated by a single injection. In such embodiments, two or more injections may be used to achieve the desired dose. In certain embodiments, a dose may be administered in two or more injections to minimize injection site reaction in an individual. In other embodiments, the compound or pharmaceutical agent is administered by infusion over an extended period of time or continuously. Doses may be stated as the amount of pharmaceutical agent per hour, day, week or month.

“Dosing regimen” is a combination of doses designed to achieve one or more desired effects.

“Double-stranded compound” means a compound comprising two oligomeric compounds that are complementary to each other and form a duplex, and wherein one of the two said oligomeric compounds comprises an oligonucleotide.

“Ectopically express” a gene means to artificially express a gene in a cell that naturally or normally does not express the gene or expresses low levels of the gene. In certain embodiments, a neuron does not normally express ASGPR or expresses low levels of ASGPR but may be manipulated ectopically express ASGPR.

“Effective amount” means the amount of compound sufficient to effectuate a desired physiological outcome in an individual in need of the compound. The effective amount may vary among individuals depending on the health and physical condition of the individual to be treated, the taxonomic group of the individuals to be treated, the formulation of the composition, assessment of the individual's medical condition, and other relevant factors.

“Efficacy” means the ability to produce a desired effect.

“Endogenously express” a gene refers to when a cell naturally or normally expresses the gene. In certain embodiments, a hepatocyte endogenously expresses ASGPR.

“Expression” includes all the functions by which a gene's coded information is converted into structures present and operating in a cell. Such structures include, but are not limited to the products of transcription and translation.

“Gapmer” means an oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.” “Hybridization” means annealing of oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases. In certain embodiments, complementary nucleic acid molecules include, but are not limited to, an antisense compound and a nucleic acid target. In certain embodiments, complementary nucleic acid molecules include, but are not limited to, an oligonucleotide and a nucleic acid target.

“Immediately adjacent” means there are no intervening elements between the immediately adjacent elements of the same kind (e.g. no intervening nucleobases between the immediately adjacent nucleobases).

“Individual” means a human or non-human animal selected for treatment or therapy.

“Inhibiting the expression or activity” refers to a reduction or blockade of the expression or activity relative to the expression of activity in an untreated or control sample and does not necessarily indicate a total elimination of expression or activity.

“Internucleoside linkage” means a group or bond that forms a covalent linkage between adjacent nucleosides in an oligonucleotide. “Modified internucleoside linkage” means any internucleoside linkage other than a naturally occurring, phosphate internucleoside linkage. Non-phosphate linkages are referred to herein as modified internucleoside linkages.

“Lengthened oligonucleotides” are those that have one or more additional nucleosides relative to an oligonucleotide disclosed herein, e.g. a parent oligonucleotide.

“Linked nucleosides” means adjacent nucleosides linked together by an internucleoside linkage.

“Mismatch” or “non-complementary” means a nucleobase of a first oligonucleotide that is not complementary to the corresponding nucleobase of a second oligonucleotide or target nucleic acid when the first and second oligonucleotides are aligned. For example, nucleobases including but not limited to a universal nucleobase, inosine, and hypoxanthine, are capable of hybridizing with at least one nucleobase but are still mismatched or non-complementary with respect to nucleobase to which it hybridized. As another example, a nucleobase of a first oligonucleotide that is not capable of hybridizing to the corresponding nucleobase of a second oligonucleotide or target nucleic acid when the first and second oligonucleotides are aligned is a mismatch or non-complementary nucleobase.

“Modulating” refers to changing or adjusting a feature in a cell, tissue, organ or organism. For example, modulating a target gene can mean to increase or decrease the level of a target gene in a cell, tissue, organ or organism. A “modulator” effects the change in the cell, tissue, organ or organism. For example, a compound can be a modulator of a target gene that decreases the amount of a target gene in a cell, tissue, organ or organism.

“MOE” means methoxyethyl.

“Monomer” refers to a single unit of an oligomer. Monomers include, but are not limited to, nucleosides and nucleotides.

“Motif” means the pattern of unmodified and/or modified sugar moieties, nucleobases, and/or internucleoside linkages, in an oligonucleotide.

“Native cell” means a cell that endogenously expresses, or endogenously expresses high levels, of a gene such as a cell surface receptor. For example, and not by limitation, a hepatocyte is a native cell for expressing asialoglycoprotein receptor (ASGPR).

“Natural” or “naturally occurring” means found in nature.

“Non-bicyclic modified sugar” or “non-bicyclic modified sugar moiety” means a modified sugar moiety that comprises a modification, such as a substituent, that does not form a bridge between two atoms of the sugar to form a second ring.

“Non-native cell” means a cell that does not endogenously express, or endogenously expresses low levels, of a gene such as a cell surface receptor. For example, a neuron is a non-native cell for expressing asialoglycoprotein receptor (ASGPR). In certain embodiments, a non-liver cell is a non-native cell for expressing asialoglycoprotein receptor (ASGPR).

“Nucleic acid” refers to molecules composed of monomeric nucleotides. A nucleic acid includes, but is not limited to, ribonucleic acids (RNA), deoxyribonucleic acids (DNA), single-stranded nucleic acids, and double-stranded nucleic acids.

“Nucleobase” means a heterocyclic moiety capable of pairing with a base of another nucleic acid. As used herein a “naturally occurring nucleobase” is adenine (A), thymine (T), cytosine (C), uracil (U), and guanine (G). A “modified nucleobase” is a naturally occurring nucleobase that is chemically modified. A “universal base” or “universal nucleobase” is a nucleobase other than a naturally occurring nucleobase and modified nucleobase, and is capable of pairing with any nucleobase.

“Nucleobase sequence” means the order of contiguous nucleobases in a nucleic acid or oligonucleotide independent of any sugar or internucleoside linkage.

“Nucleoside” means a compound comprising a nucleobase and a sugar moiety. The nucleobase and sugar moiety are each, independently, unmodified or modified. “Modified nucleoside” means a nucleoside comprising a modified nucleobase and/or a modified sugar moiety. Modified nucleosides include abasic nucleosides, which lack a nucleobase.

“Oligomeric compound” means a compound comprising a single oligonucleotide and optionally one or more additional features, such as a conjugate group or terminal group.

“Oligonucleotide” means a polymer of linked nucleosides each of which can be modified or unmodified, independent one from another. Unless otherwise indicated, oligonucleotides consist of 8-80 linked nucleosides. “Modified oligonucleotide” means an oligonucleotide, wherein at least one sugar, nucleobase, or internucleoside linkage is modified. “Unmodified oligonucleotide” means an oligonucleotide that does not comprise any sugar, nucleobase, or internucleoside modification.

“Parent oligonucleotide” means an oligonucleotide whose sequence is used as the basis of design for more oligonucleotides of similar sequence but with different lengths, motifs, and/or chemistries. The newly designed oligonucleotides may have the same or overlapping sequence as the parent oligonucleotide.

“Parenteral administration” means administration through injection or infusion. Parenteral administration includes subcutaneous administration, intravenous administration, intramuscular administration, intraarterial administration, intraperitoneal administration, or intracranial administration, e.g. intrathecal or intracerebroventricular administration.

“Pharmaceutically acceptable carrier or diluent” means any substance suitable for use in administering to an individual. For example, a pharmaceutically acceptable carrier can be a sterile aqueous solution, such as PBS or water-for-injection.

“Pharmaceutically acceptable salts” means physiologically and pharmaceutically acceptable salts of compounds, such as oligomeric compounds or oligonucleotides, i.e., salts that retain the desired biological activity of the parent compound and do not impart undesired toxicological effects thereto.

“Pharmaceutical agent” means a compound that provides a therapeutic benefit when administered to an individual.

“Pharmaceutical composition” means a mixture of substances suitable for administering to an individual. For example, a pharmaceutical composition may comprise one or more compounds or salt thereof and a sterile aqueous solution.

“Phosphorothioate linkage” means a modified phosphate linkage in which one of the non-bridging oxygen atoms is replaced with a sulfur atom. A phosphorothioate internucleoside linkage is a modified internucleoside linkage.

“Phosphorus moiety” means a group of atoms comprising a phosphorus atom. In certain embodiments, a phosphorus moiety comprises a mono-, di-, or tri-phosphate, or phosphorothioate.

“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an oligomeric compound.

“Prevent” refers to delaying or forestalling the onset, development or progression of a disease, disorder, or condition for a period of time from minutes to indefinitely.

“Prodrug” means a compound in a form outside the body which, when administered to an individual, is metabolized to another form within the body or cells thereof. In certain embodiments, the metabolized form is the active, or more active, form of the compound (e.g., drug). Typically conversion of a prodrug within the body is facilitated by the action of an enzyme(s) (e.g., endogenous or viral enzyme) or chemical(s) present in cells or tissues, and/or by physiologic conditions.

“Reduce” means to bring down to a smaller extent, size, amount, or number.

“Region” is defined as a portion of the target nucleic acid having at least one identifiable structure, function, or characteristic.

“RNAi compound” means an antisense compound that acts, at least in part, through RISC or Ago2, but not through RNase H, to modulate a target nucleic acid and/or protein encoded by a target nucleic acid. RNAi compounds include, but are not limited to double-stranded siRNA, single-stranded RNA (ssRNA), and microRNA, including microRNA mimics.

“Segments” are defined as smaller or sub-portions of regions within a nucleic acid.

“Side effects” means physiological disease and/or conditions attributable to a treatment other than the desired effects. In certain embodiments, side effects include injection site reactions, liver function test abnormalities, renal function abnormalities, liver toxicity, renal toxicity, central nervous system abnormalities, myopathies, and malaise. For example, increased aminotransferase levels in serum may indicate liver toxicity or liver function abnormality. For example, increased bilirubin may indicate liver toxicity or liver function abnormality.

“Single-stranded” in reference to a compound means the compound has only one oligonucleotide.

“Self-complementary” means an oligonucleotide that at least partially hybridizes to itself. A compound consisting of one oligonucleotide, wherein the oligonucleotide of the compound is self-complementary, is a single-stranded compound. A single-stranded compound may be capable of binding to a complementary compound to form a duplex.

“Sites,” are defined as unique nucleobase positions within a target nucleic acid.

“Specifically hybridizable” refers to an oligonucleotide having a sufficient degree of complementarity between the oligonucleotide and a target nucleic acid to induce a desired effect, while exhibiting minimal or no effects on non-target nucleic acids. In certain embodiments, specific hybridization occurs under physiological conditions.

“Specifically inhibit” a target nucleic acid means to reduce or block expression of the target nucleic acid while exhibiting fewer, minimal, or no effects on non-target nucleic acids reduction and does not necessarily indicate a total elimination of the target nucleic acid's expression.

“Standard cell assay” means assay(s) described in the Examples and reasonable variations thereof.

“Standard in vivo experiment” means the procedure(s) described in the Example(s) and reasonable variations thereof.

“Sugar moiety” means an unmodified sugar moiety or a modified sugar moiety. “Unmodified sugar moiety” or “unmodified sugar” means a 2′-OH(H) furanosyl moiety, as found in RNA (an “unmodified RNA sugar moiety”), or a 2′-H(H) moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. “Modified sugar moiety” or “modified sugar” means a modified furanosyl sugar moiety or a sugar surrogate. “Modified furanosyl sugar moiety” means a furanosyl sugar comprising a non-hydrogen substituent in place of at least one hydrogen of an unmodified sugar moiety. In certain embodiments, a modified furanosyl sugar moiety is a 2′-substituted sugar moiety. Such modified furanosyl sugar moieties include bicyclic sugars and non-bicyclic sugars.

“Sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

“Target gene” refers to a gene encoding a target.

“Targeting” means specific hybridization of a compound to a target nucleic acid in order to induce a desired effect.

“Target nucleic acid,” “target RNA,” “target RNA transcript” and “nucleic acid target” all mean a nucleic acid capable of being targeted by compounds described herein.

“Target region” means a portion of a target nucleic acid to which one or more compounds is targeted.

“Target segment” means the sequence of nucleotides of a target nucleic acid to which a compound described herein is targeted. “5′ target site” refers to the 5′-most nucleotide of a target segment. “3′ target site” refers to the 3′-most nucleotide of a target segment.

“Terminal group” means a chemical group or group of atoms that is covalently linked to a terminus of an oligonucleotide.

“Therapeutically effective amount” means an amount of a compound, pharmaceutical agent, or composition that provides a therapeutic benefit to an individual.

“Treat” refers to administering a compound or pharmaceutical composition to an individual in order to effect an alteration or improvement of a disease, disorder, or condition in the individual.

“Vector” refers to a vehicle for transferring nucleic acid to a target cell. In certain embodiments, a vector includes but is not limited to a plasmid, a virus, a virus like particle, a cassette, a lipid, a peptide, a polymer, or a particle such as a liposome or nanoparticle.

### Certain Embodiments

In certain embodiments, a method comprises contacting a non-native cell with a cell surface receptor upregulating agent, thereby generating a non-native cell ectopically expressing a cell surface receptor, and contacting the non-native cell ectopically expressing the cell surface receptor with a compound comprising a modified oligonucleotide and a conjugate group, wherein the conjugate group binds to the cell surface receptor. In certain embodiments, the non-native cell does not endogenously express the receptor or endogenously expresses the receptor at low levels relative to a native cell that endogenously expresses the receptor.

In certain embodiments, a method comprises contacting a non-native cell ectopically expressing a cell surface receptor with a compound comprising a modified oligonucleotide and a conjugate group, wherein the conjugate group binds to the cell surface receptor.

Certain embodiments are directed to and useful for ex vivo gene therapy approaches. Several clinical trials are in progress involving ex vivo gene transfer into hematopoietic stem cells (HSC), known as HSC-based gene therapy for treating a number of diseases including Wiskott-Aldrich syndrome, X-linked severe combined immunodeficiency, ß-Thalassaemia major, Adrenoleukodystrophy, and Metachromatic leukodystrophy. In these clinical trials, a lentiviral vector or gamma-retroviral vector are used for ex vivo gene transfer into HSC progenitor CD34+ cells, which are cultured and administered to the patient. Several clinical trials are in progress involving T-cell ex vivo gene therapy for cancer immunotherapy. A patient's autologous T cells are harvested from peripheral blood and harvested ex vivo, then transduced with a gene therapy vector, such as a lentivirus, to ectopically express and T-cell receptor specific to a cancer antigen. The T-cells ectopically expressing the receptor against the cancer antigen are then administered to the patient. Another recent approach involves gene therapy transfer of chimeric antigen receptors (CAR) rather than T-cell receptors to T cells ex vivo, and infusion back to the patient. Successful CAR clinical trials have been reported in B-cell cancer patients.

In certain embodiments useful for ex vivo gene therapy, the non-native cell is contacted with a the cell surface receptor upregulating agent ex vivo. In certain embodiments, a non-native cell, such as a non-liver cell (e.g. a neuron or eye cell), is contacted ex vivo with a cell surface receptor upregulating agent, which can be a vector, such as a lentivirus or adeno-associated virus (AAV), comprising a nucleic acid encoding asialoglycoprotein receptor (ASGPR) or the ASGPR major subunit (ASGPR1). In certain embodiments, the non-native cell ectopically expressing the cell surface receptor, such as ASGPR or ASGPR1, can be administered to a subject and the subject can be administered the compound comprising a modified oligonucleotide and a conjugate group, such as GalNAc, that binds to the receptor. In certain embodiments, the non-native cell ectopically expressing the cell surface receptor, such as ASGPR or ASGPR1, can be contacted ex vivo with the compound comprising a modified oligonucleotide and a conjugate group, such as GalNAc, that binds to the receptor. Subsequently, the non-native cell can be administered to the subject In certain embodiments, the cell surface receptor upregulating agent comprises a vector and a nucleic acid encoding the cell surface receptor. In certain embodiments, the vector comprises a virus. In certain embodiments, the virus is suitable for gene therapy. In certain embodiments, the virus is a lentivirus, gamma retrovirus, adenovirus, or AAV. In certain embodiments, the cell surface receptor upregulating agent is a modified oligonucleotide complementary to a target site within a translation suppression element region of a RNA transcript encoding a cell surface receptor. In certain embodiments, the cell surface receptor upregulating agent is a RNA transcript encoding a cell surface receptor. In certain embodiments, the cell surface receptor upregulating agent is a CRISPR system homology directed repair insertion cassette comprising a nucleic acid encoding a cell surface receptor. In certain embodiments, the cell surface receptor upregulating agent is a vector comprising a nucleic acid encoding a Cas9 activator system comprising a nuclease-defective Cas9 (dCas9) and a transactivator capable of transcribing a cell surface receptor. In certain embodiments, the cell surface receptor upregulating agent is a vector comprising a nucleic acid encoding a CRISPR associated nuclease activator system comprising a nuclease-defective CRISPR associated nuclease, such as a nuclease-defective Cpf1 or dCas9, and a transactivator capable of transcribing a cell surface receptor.

Certain embodiments are useful for in vivo gene therapy approaches. In certain embodiments, the non-native cell is contacted with the cell surface receptor upregulating agent in vivo. In certain embodiments, a non-native cell, such as a non-liver cell (e.g. a neuron or eye cell), is contacted in vivo with the cell surface receptor upregulating agent, which can be a vector, such as a lentivirus or adeno-associated virus (AAV), comprising a nucleic acid encoding asialoglycoprotein receptor (ASGPR) or the ASGPR major subunit (ASGPR1). In certain embodiments, the non-native cell ectopically expressing the cell surface receptor, such as ASGPR or ASGPR1, can be contacted in vivo with the compound comprising a modified oligonucleotide and a conjugate group, such as GalNAc, that binds to the receptor. In certain embodiments, the cell surface receptor upregulating agent comprises a vector and a nucleic acid encoding the cell surface receptor. In certain embodiments, the vector comprises a virus. In certain embodiments, the virus is suitable for gene therapy. In certain embodiments, the virus is a lentivirus, gamma retrovirus, adenovirus, or AAV. In certain embodiments, the cell surface receptor upregulating agent is a modified oligonucleotide complementary to a target site within a translation suppression element region of a RNA transcript encoding a cell surface receptor. In certain embodiments, the cell surface receptor upregulating agent is a RNA transcript encoding a cell surface receptor. In certain embodiments, the cell surface receptor upregulating agent is a CRISPR system homology directed repair insertion cassette comprising a nucleic acid encoding a cell surface receptor. In certain embodiments, the cell surface receptor upregulating agent is a vector comprising a nucleic acid encoding a Cas9 activator system comprising a nuclease-defective Cas9 (dCas9) and a transactivator capable of transcribing a cell surface receptor. In certain embodiments, the cell surface receptor upregulating agent is a vector comprising a nucleic acid encoding a CRISPR associated nuclease activator system comprising a nuclease-defective CRISPR associated nuclease, such as a nuclease-defective Cpf1 or dCas9, and a transactivator capable of transcribing a cell surface receptor. In certain embodiments, a method of delivering a compound to a subject ectopically expressing a cell surface receptor in a non-native cell comprises administering to the subject a cell surface receptor upregulating agent, thereby generating a non-native cell ectopically expressing the cell surface receptor in the subject; and administering to the subject a compound comprising a modified oligonucleotide and a conjugate group, wherein the conjugate group binds to the receptor, thereby delivering the compound to a non-native cell ectopically expressing the cell surface receptor in the subject. In certain embodiments, the cell surface receptor upregulating agent comprises a vector and a nucleic acid encoding the cell surface receptor. In certain embodiments, the vector comprises a virus. In certain embodiments, the virus is suitable for gene therapy. In certain embodiments, the virus is a lentivirus, gamma retrovirus, adenovirus, or AAV. In certain embodiments, the cell surface receptor upregulating agent is a modified oligonucleotide complementary to a target site within a translation suppression element region of a RNA transcript encoding a cell surface receptor. In certain embodiments, the cell surface receptor upregulating agent is a RNA transcript encoding a cell surface receptor. In certain embodiments, the cell surface receptor upregulating agent is a CRISPR system homology directed repair insertion cassette comprising a nucleic acid encoding a cell surface receptor. In certain embodiments, the cell surface receptor upregulating agent is a vector comprising a nucleic acid encoding a Cas9 activator system comprising a nuclease-defective Cas9 (dCas9) and a transactivator capable of transcribing a cell surface receptor. In certain embodiments, the cell surface receptor upregulating agent is a vector comprising a nucleic acid encoding a CRISPR associated nuclease activator system comprising a nuclease-defective CRISPR associated nuclease, such as a nuclease-defective Cpf1 or dCas9, and a transactivator capable of transcribing a cell surface receptor.

In certain embodiments, the cell surface receptor is asialoglycoprotein receptor (ASGPR) or the ASGPR major subunit (ASGPR1). In certain embodiments, the conjugate group is GalNAc. In certain embodiments, the non-native cell is a non-liver cell, such as a neuron or eye cell.

In certain embodiments, a method of delivering a compound to a subject ectopically expressing a cell surface receptor in non-native cells comprises administering to the subject a compound comprising a modified oligonucleotide and a conjugate group, wherein the conjugate group binds to the cell surface receptor. In certain embodiments, the cell surface receptor is asialoglycoprotein receptor (ASGPR) or the ASGPR major subunit (ASGPR1). In certain embodiments, the conjugate group is GalNAc. In certain embodiments, the non-native cell is a non-liver cell, such as a neuron or eye cell.

In any of the aforementioned embodiments, the non-native cell can be a non-liver cell, such as an eye, muscle, heart, skin, kidney, lung, pancreas, intestinal, fat, spleen, bone, testes, ovary, pituitary, immune, or bladder cell. In any of the aforementioned embodiments, the non-native cell can be a cell in the central nervous system (CNS), such as a brain cell, which can be a neuron.

In any of the aforementioned embodiments, the receptor can be a liver cell receptor, such as asialoglycoprotein receptor (ASGPR). In certain embodiments, the ASGPR is the ASGPR major subunit (ASGPR1).

In any of the aforementioned embodiments, the compound contacted with the non-native cell can comprise a modified oligonucleotide and a conjugate group, wherein the conjugate group comprises N-acetyl galactosamine (GalNAc).

In any of the aforementioned embodiments, the conjugate group can comprise:

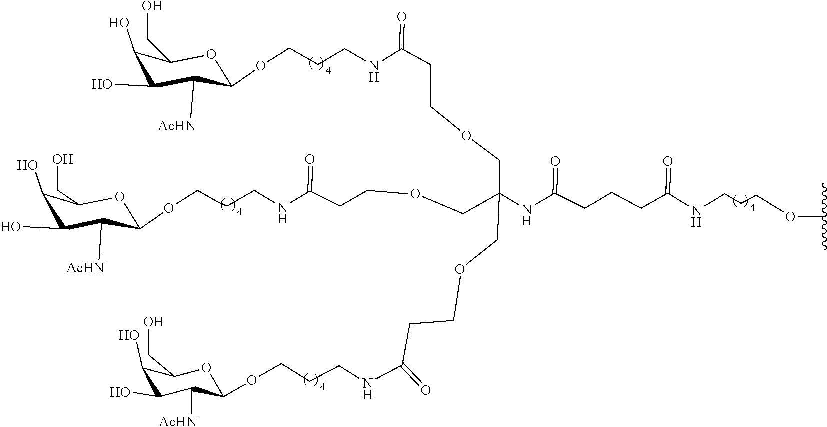

In any of the aforementioned embodiments, the compound can be single-stranded.

In any of the aforementioned embodiments, the compound can be double-stranded.

In any of the aforementioned embodiments, the modified oligonucleotide can be 12 to 30 linked nucleosides in length.

In any of the aforementioned embodiments, the modified oligonucleotide can comprise at least one modified internucleoside linkage, at least one modified sugar moiety, or at least one modified nucleobase.

In any of the aforementioned embodiments, at least one modified sugar comprises a 2′-O-methyoxyethyl or a bicyclic sugar can be selected from the group consisting of: 4′-(CH2)-O-2′ (LNA); 4′-(CH2)2-O-2′ (ENA); and 4′-CH(CH3)-O-2′ (cEt).

In any of the aforementioned embodiments, each modified internucleoside can be a phosphorothioate linkage.

In any of the aforementioned embodiments, each cytosine can be a 5-methylcytosine.

In any of the aforementioned embodiments, the modified oligonucleotide can comprise:

a gap segment consisting of linked deoxynucleosides;

a 5′ wing segment consisting of linked nucleosides;

a 3′ wing segment consisting linked nucleosides;

wherein the gap segment is positioned immediately adjacent to and between the 5′ wing segment and the 3′ wing segment and wherein each nucleoside of each wing segment comprises a modified sugar.

In any of the aforementioned embodiments, the compound can comprise ribonucleotides.

In any of the aforementioned embodiments, the compound can comprise deoxyribonucleotides.

### Certain Cell Surface Receptor Upregulating Agents

A. Certain Vectors

Any suitable gene therapy vector known in the art may be used to ectopically express a receptor, such as ASGPR or ASGPR1, in a non-native cell. Examples of gene therapy vectors suitable for use in certain embodiments provided herein include, but are not limited to, adenovirus vectors, Adeno-associated virus (AAV) vectors, HSV-1 vectors, and lentivirus vectors. Below is a non-limiting description of suitable vectors and references cited are incorporated by reference in their entireties herein.

1. Adenovirus Vectors

Adenoviruses (Ads) have a genome encoding approximately 35 proteins that are expressed in an early phase and late phase occurring before and after viral DNA replication, respectively. The early genes encode proteins that control viral DNA replication and the late genes encode structural proteins). Ads vinons lyse infected cells to release infectious virus.

There are 57 serotypes of human Ads, Ad1-Ad57, that comprise seven species A-G. Most Ad vectors are replication-defective (RD) or replication-competent (RC) genetically modified versions of Ad5. In certain embodiments, the vector is a replication defective adenovirus in which the essential E1A and E1B genes are deleted and replaced by an expression cassette comprising a promoter which drives ectopic expression of a receptor in a non-native cell. An example of a suitable promoter includes, but is not limited to, the cytomegalovirus immediate early (CMV) promoter. In certain embodiments, the adenovirus vector lacks the E3 genes. Construction and preparation of adenovirus vectors are described, for example, in Brunetti-Pierri N, Ng P. 2011 Hum Mol Genet; 20:7-13, which is incorporated by reference in its entirety herein.

In certain embodiments, the vector is a helper-dependent (HDAd) replication defective adenovirus vector that has most of the genome deleted but retains genes required for virion packaging. Construction and preparation of HDAd are described in Parks R J et al. 1996 PNAS 93:13565-13570, which is incorporated by reference in its entirety herein.

In certain embodiments, a vector comprising a nucleic acid encoding the cell surface receptor is an adenovirus vector. In certain embodiments, an adenovirus vector can comprise a nucleic acid encoding ASGPR or ASGPR1. In certain embodiments, an adenovirus vector is contacted with a non-liver cell, such as a brain, eye, muscle, heart, skin, kidney, lung, pancreas, intestinal, fat, spleen, bone, testes, ovary, pituitary, immune, or bladder cell.

2. Adeno-Associated Virus (AAV) Vectors Adeno-associated virus (AAV) is a small non-pathogenic virus belonging to the parvoviridae family that is commonly used as a gene therapy vector. AAV vectors are attractive for gene therapy because they are considered non-pathogenic and cause only mild immune responses. Positive human clinical trials using AAV gene therapy vectors have been reported.

At least twelve human serotypes of AAV (AAV1-12) have been identified, with serotype 2 (AAV2) being the most extensively studied. It has been reported that AAV2 has tropism towards skeletal muscle, neurons, vascular smooth muscle cells, and hepatocytes. In clinical trials, AAV2 vectors have been delivered to the brain by intracranial administration. AAV6 has been reported to be effective in infecting airway epithelial cells. AAV1, AAV5, and AAV7 have been reported to be effective in transducing skeletal muscle cells. AAV8 has been reported to be effective in transducing hepatocytes. AAV1 and AAV5 have been reported to be effective in transducing vascular endothelial cells. Most AAV serotypes are been reported to have neuron tropism. AAV5 has been reported to transduce astrocytes. Design of various AAV vectors useful for gene therapy have been described in Gao G P et al., PNAS 2003: 99(18): 11854-9; Halbert C L et al., J. Virol. 2001: 75(14): 6615-24; Rabinowitz J E et al., J. Virol. 2004: 75(14): 6615-24; Chen S et al., 2005 Human Gene Therapy 16(2): 235-47; and Bouard D et al., Br J Pharmacol 2009: 157(2): 153-165; each of which is incorporated by reference in its entirety herein. Design and CNS administration of AAV9 vectors has been described in Meyer K et al., 2015 Molecular Therapy vol. 23 no. 3, 477-487, which is incorporated by reference in its entirety herein. Design and CNS administration of AAV6 vectors has been described in Kaplan A et al., 2014 Neuron (81): 333-348, which is incorporated by reference in its entirety herein. Design and CNS administration of AAV8 vectors has been described in Passini M A et al., 2010 JCI vol. 120(4): 1253-64, which is incorporated by reference in its entirety herein. Design and CNS administration of AAV2/8 and AAV2/9 vectors has been described in Chakrabarty P et al., 2013 PLOS One Vol. 8(6):e67680, which is incorporated by reference in its entirety herein.

In certain embodiments, a vector comprising a nucleic acid encoding the cell surface receptor is an AAV vector. In certain embodiments, the AAV vector is AAV1, AAV2, AAV3, AAV4, AAV5, AAV6, AAV7, AAV8, AAV9, AAV10, AAV11, or AAV12. In certain embodiments, the AAV vector is AAV2.

In certain embodiments, the AAV vector is AAV9. In certain embodiments, AAV9, AAV2, or a rationally engineered capsid AAV2.5, which is a hybrid of AAV2 and AAV1 that incorporates six amino acids from AAV1 into the AAV2 capsid, can be used to ectopically express a receptor in a non-native cell. AAV9, AAV2, and AAV2.5 vectors are described in Gray S J et al., Gene Therapy (2013) 20, 450-459, which is incorporated by reference in its entirety herein. AAV2 vectors are described in PCT application publications WO 2015/121501, WO 2012/158757, WO 2010/093784, and WO/2016/044478, each of which is incorporated by reference in its entirety herein. In certain embodiments, any of the aforementioned AAV vectors can comprise a nucleic acid encoding ASGPR or ASGPR1. In certain embodiments, an AAV vector is contacted with a non-liver cell, such as a brain, eye, muscle, heart, skin, kidney, lung, pancreas, intestinal, fat, spleen, bone, testes, ovary, pituitary, immune, or bladder cell.

3. HSV Vectors

Herpes simplex virus (HSV), such as HSV-1, is an enveloped virus having a 152 kb double-stranded DNA genome encoding over 80 genes, and infects many cell types including neurons and glia cells. HSV vectors can be recombinant virus vectors (RV) and amplicon vectors. In certain embodiments, a vector comprising a nucleic acid encoding the cell surface receptor is an HSV vector. In certain embodiments, an HSV vector can be an amplicon, replication-defective, or replication-competent vector.

HSV amplicon vectors are plasmid-derived vectors containing the origin of replication (ori) and HSV cleavage-packaging recognition sequences (pac). HSV amplicon vectors can be produced by infection with defective helper HSVs or transfection of HSV genes. In certain embodiments, a HSV vector is a HVS amplicon vector. HSV amplicon vector production is described in Epstein A L, 2009 Mem Inst Oswaldo Cruz; 104:399-410, which is incorporated by reference in its entirety herein.

Replication-defective HSV vectors have deletions in one or more genes essential for the lytic cycle. In certain embodiments, the HSV vector is a replication-defective vector. Replication-defective HSV vectors suitable for use in embodiments provided herein include, but are not limited to, those described in Burton E A et al., 2002 Curr Opin Biotechnol. 13:424-8; Berto E et al., 2005 Gene Ther. 12(Suppl 1):S98-102; and Krisky D M et al., 1998 Gene Ther. 5:1517-30; each of which is incorporated by reference in its entirety herein. Replication-defective HSV vectors have been shown preclinically to be useful in treating epilepsy, multiple sclerosis, Alzheimer Disease, and Parkinson's disease.

In certain embodiments, a HSV vector is contacted with a brain cell. HSV is neurotropic and can be rationally designed for gene therapy treatment of neurological diseases. Design of HSV vectors for neurological applications is described in Frampton A R et al., 2005 Gene Ther. 12:891-901 and Palmer J A et al., 2000 J. Virol. 74:5604-18, each of which is incorporated by reference in its entirety herein.

In certain embodiments, any of the aforementioned HSV vectors can comprise a nucleic acid encoding ASGPR or ASGPR1. In certain embodiments, a HSV vector is contacted with a non-liver cell, such as a brain, eye, muscle, heart, skin, kidney, lung, pancreas, intestinal, fat, spleen, bone, testes, ovary, pituitary, immune, or bladder cell.

4. Lentivirus Vectors

Lentiviruses belong to the retroviridae family that includes HIV, SIV, FIV, EIAV, and Visna, characterized by along incubation period. Lentiviral vectors are capable of integrating into the host genome of nondividing cells, thereby providing the potential for stable ectopic expression of a cell surface receptor in neurons, for example. Positive results have been reported for intracerebral administration of lentivirus vectors. It has been reported that intrastriatal injection of a lentivirus vector resulted in higher neuronal transduction than observed for AAV, adenovirus, and retrovirus vectors.

Lentivirus vector design and production is described in Connolly J B, 2002 Gene Therapy 9(24):1730-34, which is incorporated by reference in its entirety herein. By way of example and not limitation, a producer cell line can be transfected with (i) a plasmid comprising the surface receptor and lentiviral long terminal repeats (LTRs) for host cell integration; (ii) a plasmid encoding the gag and pol viral structural genes, and (iii) plasmid encoding envelope protein.

In certain embodiments, a vector comprising a nucleic acid encoding the cell surface receptor is a lentivirus vector. In certain embodiments, a lentivirus vector can comprise a nucleic acid encoding ASGPR or ASGPR1. In certain embodiments, a lentivirus vector is contacted with a non-liver cell, such as a brain, eye, muscle, heart, skin, kidney, lung, pancreas, intestinal, fat, spleen, bone, testes, ovary, pituitary, immune, or bladder cell.

5. Non-Viral Vectors

In certain embodiments, the vector comprising a nucleic acid encoding a cell surface receptor is a non-viral gene delivery systems including but not limited to plasmids, expression cassettes, virus like particles, nanoparticles, liposomes, cationic lipids, and polycationic polymers. In certain embodiments, a cationic polypeptide vector, such as polylysine or spermidine, can bind to the nucleic acid encoding a cell surface receptor. In certain embodiments, a cationic lipid vector can encapsulate nucleic acid encoding a cell surface receptor in a liposome that enters the non-native cell by endocytosis. In any of the aforementioned non-viral vectors, the vector can comprise or consist of nucleic acid encoding ASGPR or ASGPR1.

6. Non-Specific and Tissue-Specific Promoters

In any of the embodiments described herein, the vector can comprise an expression cassette comprising a promoter and a nucleic acid encoding a cell surface receptor.

In any of the embodiments described herein, the vector can comprise a non-specific promoter to drive ectopic expression of the cell surface protein in any cell the vector contacts. Examples of non-specific promoters include, but are not limited to, the cytomegalovirus (CMV) promoter, Rous sarcoma virus (RSV) promoter, simian virus 40 (SV40) promoter, and mammalian elongation factor 1α (EF1α) promoter.

In any of the embodiments described herein, the vector can comprise a tissue specific promoter to drive ectopic expression of the cell surface protein in a tissue-specific manner. Examples of tissue-specific promoters include, but are not limited to, neuron-specific promoters such as human synapsin 1 (hSYN1) promoter; eye-specific promoters such as vitelliform macular dystrophy (VMD2) promoter, rhodopsin promoter, opsin promoter, or thymocyte antigen promoter. Any tissue-specific promoter known in the art can be used. The Tissue Specific Promoter Database (TiProD) is a catalog of tissue-specific promoters and any desired promoter can be selected from the database and used in vectors for tissue-specific ectopic expression of a surface protein as described in embodiments herein. In certain embodiments, tissue-specific promotors known in the art can be used for tissue-specific expression of a surface protein in the liver, brain, eye, muscle, heart, skin, kidney, lung, pancreas, intestinal, fat, spleen, bone, testes, ovary, pituitary, immune, or bladder cell.

B. Compounds Targeting Cell Surface Receptor uORF

Translation of a polypeptide or protein encoded by an mRNA typically begins at the start codon of the primary open reading frame (pORF) of the mRNA. Some mRNA transcripts also comprise one or more additional start codons. Such additional start codons may be upstream of the pORF start codon. Such an additional start codon that is upstream of a pORF is referred to as an upstream open reading frame (uORF) start site. The potential role of additional start sites in regulating translation of pORF protein products has been discussed previously (see Barbosa et al. PLOS Genetics. 9, e1003529 (2013)) which is hereby incorporated by reference in its entirety. Mutations that introduce or eliminate an additional start codon (a uORF start codon) in a transcript can disrupt regulation of its translation and can lead to disease (see Calvo et al. Proc. Natl. Acad. Sci. 106, 7507 (2009)) which is hereby incorporated by reference in its entirety.

In certain embodiments, a cell surface receptor upregulating agent is a compound that interacts with the 5′-UTR of a transcript encoding a cell surface receptor to increase translation of the cell surface receptor. For example, in certain embodiments, a cell surface receptor upregulating agent is a compound targeted to one or more regions of the 5′-UTR. These regions of the 5′-UTR may include a translation suppression element, such as a stem-loop structure or a uORF. When the compound, such as an antisense compound, interacts with the translation suppression element in the 5′-UTR, the antisense compound increases translation of the transcript encoding the cell surface receptor. One aspect of the invention is the increase in expression through contacting a cell with an agent targets a translation suppression element in the 5′-UTR. In certain embodiments, antisense compounds targeted to the 5′-UTR increase expression of a given target protein by disrupting a translation suppression element within the 5′-UTR.

Antisense oligonucleotide technology has been used most often to reduce the amount an mRNA via antisense induced RNase H cleavage or to alter splicing of a pre-mRNA transcript in a cell. In certain embodiments, an increase in the expression of a cell surface receptor in a cell is achieved by having the antisense compound reduce ribosomal recognition of one or more upstream open reading frames. In certain embodiments, recognition of an upstream open reading frame reduces expression of a cell surface receptor in a cell. Therefore, in certain embodiments, targeting the upstream open reading frame, or the nucleobase sequence upstream or downstream of the upstream open reading frame, reduces ribosomal recognition of the upstream open reading frame and thereby increases expression of one or more cell surface receptors. Therefore, in certain embodiments, targeting the upstream open reading frame, or the nucleobase sequence upstream or downstream of the upstream open reading frame, reduces ribosomal recognition of the upstream open reading frame and thereby increases ribosomal recognition of a start codon in the primary open reading frame.

In certain embodiments, antisense compounds are provided to increase expression of a cell surface receptor. In certain instances, a transcript encoding a cell surface receptor includes a pORF and one or more additional start sites, such as uORF start sites. In certain embodiments, modified oligonucleotides complementary to the cell surface receptor transcript at or near such uORF start sites are provided. Antisense compounds designed to reduce the amount of a target protein typically induce cleavage of the target transcript (e.g., through recruitment of RNase H). In contrast, in certain embodiments of the present invention, modified oligonucleotides are not designed to elicit cleavage. Rather, in certain such embodiments, the modified oligonucleotides mask a uORF start site in favor of increased translation at a pORF start site. In certain embodiments, the modified oligonucleotides of the disrupt initiation of translation at a uORF start site and, in certain embodiments, thereby increase translation of the cell surface receptor. In certain embodiments, modified oligonucleotides disrupt the regulatory function of the 5′-UTR. In certain such embodiments, translation of the desired cell surface receptor is increased. In certain embodiments, modified oligonucleotides recruit proteins to the transcript that interfere with initiation of translation at the uORF start site. In certain embodiments, antisense compounds result in decreased translation of a uORF polypeptide.

C. CRISPR System

In certain embodiments, the cell surface receptor upregulating agent is a CRISPR system homology directed repair insertion cassette comprising a nucleic acid encoding a cell surface receptor. Use of Cluster Regulatory Interspaced Short Palindromic Repeats (CRISPR) to edit or disable genes has been described. See for example Jinek et al., Scinece 337: 816-821 (2012); Mali et al. Science 339: 823-826 (2013).

In certain embodiments, a non-native cell is engineered to express Cas9, a crRNA, and a tracrRNA, such as by contacting the non-native cell with one or more vectors comprising nucleic acid encoding for Cas9, the crRNA, and the tracrRNA separately or together. In certain embodiments, a non-native cell is engineered to express Cas9 and a tracrRNA, and contacted with a modified crRNA. In certain embodiments, a non-native cell is engineered to express Cas9 and contacted with a modified crRNA. Consequently, Cas9 will cleave the genome of the non-native cell at the locus of interest and the non-native cell is contacted with homology directed repair insertion cassette comprising a nucleic acid encoding a cell surface receptor, such as ASGPR or ASGPR1, thereby generating a non-native cell ectopically expressing the cell surface receptor. This methodology is described in Zheng Q et al., 2014 BioTechniques, Vol. 57, No. 3, pp. 115-124, which is incorporated by reference in its entirety herein.

In certain embodiments, a non-native cell is engineered to express a CRISPR associated nuclease, such as Cas9 or Cpf1, a crRNA, and a tracrRNA, such as by contacting the non-native cell with one or more vectors comprising nucleic acid encoding for a CRISPR associated nuclease, the crRNA, and the tracrRNA separately or together. In certain embodiments, a non-native cell is engineered to express a CRISPR associated nuclease and a tracrRNA, and contacted with a modified crRNA. In certain embodiments, a non-native cell is engineered to express a CRISPR associated nuclease and contacted with a modified crRNA. Consequently, the CRISPR associated nuclease will cleave the genome of the non-native cell at the locus of interest and the non-native cell is contacted with homology directed repair insertion cassette comprising a nucleic acid encoding a cell surface receptor, such as ASGPR or ASGPR1, thereby generating a non-native cell ectopically expressing the cell surface receptor.

In certain embodiments, the cell surface receptor upregulating agent is a vector comprising a nucleic acid encoding a Cas9 activator system comprising a nuclease-defective Cas9 (dCas9) and a transactivator capable of transcribing a cell surface receptor. Several systems have been generated to provide dCas9 the ability to activate gene expression, including dCas9-VP64, dCas9-VPR, dCas9-SAM, and dCas9-Suntag. Chavez A et al., 2016 Nat Methods 13(7):563-7, which is incorporated by reference in its entirety herein. In certain embodiments, a non-native cell is engineered to express a crRNA directed to a cell surface receptor, a tracrRNA, and a nuclease-defective Cas9 (dCas9) linked to a transactivator capable of transcribing the cell surface receptor, thereby inducing the non-native cell to endogenously express the cell surface receptor. In certain embodiments, the transactivator is selected from the group consisting of dCas9-VP64, dCas9-VPR, dCas9-SAM. In certain embodiments, a non-native cell is contacted with a modified crRNA directed to a cell surface receptor, a tracrRNA, and a nuclease-defective Cas9 (dCas9) linked to a transactivator capable of transcribing the cell surface receptor, thereby inducing the non-native cell to endogenously express the cell surface receptor. In certain embodiments, a non-native cell is contacted with a modified crRNA directed to a cell surface receptor and a nuclease-defective Cas9 (dCas9) linked to a transactivator capable of transcribing the cell surface receptor, thereby inducing the non-native cell to endogenously express the cell surface receptor.

In certain embodiments, the cell surface receptor upregulating agent is a vector comprising a nucleic acid encoding a CRISPR associated nuclease activator system comprising a nuclease-defective CRISPR associated nuclease and a transactivator capable of transcribing a cell surface receptor. In certain embodiments, a non-native cell is engineered to express a crRNA directed to a cell surface receptor, a tracrRNA, and a nuclease-defective CRISPR associated nuclease linked to a transactivator capable of transcribing the cell surface receptor, thereby inducing the non-native cell to endogenously express the cell surface receptor. In certain embodiments, the transactivator is selected from the group consisting of a CRISPR associated nuclease linked to VP64, dCas9-VPR, dCas9-SAM. In certain embodiments, a non-native cell is contacted with a modified crRNA directed to a cell surface receptor, a tracrRNA, and a nuclease-defective CRISPR associated nuclease linked to a transactivator capable of transcribing the cell surface receptor, thereby inducing the non-native cell to endogenously express the cell surface receptor. In certain embodiments, a non-native cell is contacted with a modified crRNA directed to a cell surface receptor and a nuclease-defective CRISPR associated nuclease linked to a transactivator capable of transcribing the cell surface receptor, thereby inducing the non-native cell to endogenously express the cell surface receptor. In certain embodiments, the CRISPR associated nuclease is Cpf1.

In any of the foregoing embodiments, the modified crRNA can have improved stability relative to unmodified crRNA. In certain embodiments, modified crRNA is stabilized at the 5′ end and/or the 3′. In certain embodiments, such stabilized crRNA is resistant to exonuclease and/or endonuclease digestion. In certain embodiments, modified crRNA have improved affinity for target DNA relative to unmodified crRNA. In certain embodiments, modified crRNA have improved selectivity for target DNA relative to unmodified crRNA. In certain embodiments, modified crRNA have improved affinity for tracrRNA relative to unmodified crRNA. In certain embodiments, modified crRNA have improved cellular uptake relative to unmodified crRNA.

In certain such embodiments, the modifications increase affinity for the target DNA allowing the modified crRNA to be shortened while retaining sufficient affinity to hybridize to target DNA and to tracrRNA. Thus, in certain embodiments, modified crRNA is shorter than unmodified crRNA. In certain embodiments, modified crRNA is 40-50 linked nucleosides in length. In certain embodiments, modified crRNA is 35-45 linked nucleosides in length. In certain embodiments, modified crRNA is 30-40 linked nucleosides in length. In certain embodiments, modified crRNA is 25-35 linked nucleosides in length. In certain embodiments, modified crRNA is 20-30 linked nucleosides in length. In certain embodiments, modified crRNA is 25-35 linked nucleosides in length. In certain embodiments, modified crRNA is 20-30 linked nucleosides in length. In certain such embodiments, such shorter crRNA have improved uptake properties. In certain embodiments, modified crRNA are taken into cells without transfection reagents or electroporation. In certain such embodiments, the cells are in an animal. In certain embodiments, the animal expresses Cas9. In certain embodiments, the animal is previously or concomitantly treated with a means of expressing Cas9. In certain such embodiments, such treatment comprises administration of a vector for delivering Cas9. In certain such embodiments, such vector is a viral vector, for example adeno-associated virus (AAV). In certain such embodiments, the viral vector expresses a S. aureus derived Cas9 that fits into an AAV vector.

The present invention also provides modified oligonucleotides for use as scrRNA in CRISPR systems. As used herein, “scrRNA” or “single crRNA” means an oligonucleotide that comprises a scrRNA target recognition portion and a nuclease recognition portion and does not comprise a tracrRNA recognition portion or a tracrRNA. In certain embodiments, scrRNAs comprise a self-complementary region. In certain such embodiments, the nuclease recognition portion partially or completely overlaps with the self-complementary region. As used herein, “scrRNA target recognition portion” is a portion of an oligonucleotide with a nucleobase sequence that is complementary to a scrRNA DNA target. As used herein, “nuclease recognition portion” is a portion of an oligonucleotide that can bind to, associate with, or contribute to the binding to or association with a nuclease that is not a Cas9 nuclease. In certain embodiments, the nuclease recognition portion of an oligonucleotide binds to or associates with a Cpf1 nuclease. In certain embodiments, such modified scrRNA have improved stability relative to unmodified scrRNA. In certain embodiments, modified scrRNA is stabilized at the 5′ end and/or the 3′. In certain embodiments, such stabilized scrRNA is resistant to exonuclease and/or endonucleoase digestion. In certain embodiments, modified scrRNA have improved affinity for scrRNA target DNA relative to unmodified scrRNA. In certain embodiments, modified scrRNA have improved selectivity for scrRNA target DNA relative to unmodified scrRNA. In certain embodiments, modified scrRNA have improved affinity for a nuclease relative to unmodified scrRNA. In certain embodiments, modified scrRNA have improved cellular uptake relative to unmodified scrRNA.

In certain such embodiments, the modifications increase affinity for the scrRNA target DNA allowing the modified scrRNA to be shortened while retaining sufficient affinity to hybridize to scrRNA target DNA and a nuclease. Thus, in certain embodiments, modified scrRNA is shorter than unmodified scrRNA. In certain embodiments, modified scrRNA is 40-50 linked nucleosides in length. In certain embodiments, modified scrRNA is 35-45 linked nucleosides in length. In certain embodiments, modified scrRNA is 30-40 linked nucleosides in length. In certain embodiments, modified scrRNA is 25-35 linked nucleosides in length. In certain embodiments, modified scrRNA is 20-30 linked nucleosides in length. In certain embodiments, modified scrRNA is 25-35 linked nucleosides in length. In certain such embodiments, such shorter scrRNA have improved uptake properties. In certain embodiments, modified scrRNA are taken into cells without transfection reagents or electroporation. In certain such embodiments, the cells are in an animal. In certain embodiments, the animal expresses a nuclease that is recognized by the scrRNA (e.g., a Cpf1 nuclease). In certain embodiments, the animal is previously or concomitantly treated with a means of expressing a nuclease that is recognized by the scrRNA (e.g., a Cpf1 nuclease). In certain such embodiments, such treatment comprises administration of a vector for delivering a nuclease that is recognized by the scrRNA (e.g., a Cpf1 nuclease). In certain such embodiments, such vector is a viral vector, for example adeno-associated virus (AAV).

In certain embodiments, the modified crRNA has a modification selected from the table below:

TABLE A
crRNA modification motifs

| 29-mers | 42-mers |
|---|---|
| f₇r₆kr₃kr₃kr₃krk₂ | f₁₀r₁₈kr₄kr₂kr₃k₂ |
| mf₆r₆kr₃kr₃kr₃krk₂ | mf₉r₁₈kr₄kr₂kr₃k₂ |
| mf₆r₁₀k₆r₂k₄ | mr₂₇kr₄kr₂kr₃k₂ |
| mr₁₆k₆r₂k₄ | mr₉f₁₀k₆r₂kr₄kr₂kr₃k₂ |
| mr₆f₁₀k₆r₂k₄ | mr₉f₁₀l₆r₂kr₄kr₂kr₃k₂ |
| mf₆r₁₀f₆r₂k₄ | mr₉f₁₆r₂kr₄kr₂kr₃k₂ |
| mf₆r₁₀l₆r₂k₄ | mr₃₂kr₂kr₃k₂ |
| mr₆f₁₀l₆r₂k₄ | ef₉r₁₈kr₄kr₂kr₃k₂ |
| mf₆r₁₀k₆r₂l₄ | r(MOP)f₉r₁₈kr₄kr₂kr₃k₂ |
| mr₁₆k₆r₂l₄ | d(MOP)f₉r₁₈kr₄kr₂kr₃k₂ |
| mr₆f₁₀k₆r₂l₄ | f(MOP)f₉r₁₈kr₄kr₂kr₃k₂ |
| mf₆r₁₀f₆r₂l₄ | r(MP)f₉r₁₈kr₄kr₂kr₃k₂ |
| r(MOP)f₆r₆kr₃kr₃kr₃krk₂ | d(MP)f₉r₁₈kr₄kr₂kr₃k₂ |
| d(MOP)f₆r₆kr₃kr₃kr₃krk₂ | f(MP)f₉r₁₈kr₄kr₂kr₃k₂ |
| f(MOP)f₆r₆kr₃kr₃kr₃krk₂ | r(MMI)f₉r₁₈kr₄kr₂kr₃k₂ |
| r(MP)f₆r₆kr₃kr₃kr₃krk₂ | d(MMI)f₉r₁₈kr₄kr₂kr₃k₂ |
| d(MP)f₆r₆kr₃kr₃kr₃krk₂ | f(MMI)f₉r₁₈kr₄kr₂kr₃k₂ |
| f(MP)f₆r₆kr₃kr₃kr₃krk₂ | mr₃₂kr₂k(G-Clamp)r₂k₂ |
| r(MOP)f₆r₁₀k₆r₂k₄ | mr₂₇k₃r₂kr₂kr₃k₂ |
| d(MOP)f₆r₁₀k₆r₂k₄ | mf₉r₁₈k₃r₂kr₂kr₃k₂ |
| f(MOP)f₆r₁₀k₆r₂k₄ | mf₉r₁₁ (5-Propyne-U)₄r₃k₃r₂kr₂kr₃k₂ |
| r(MP)f₆r₁₀k₆r₂k₄ | 29-mers |
| r(MP)f₆r₁₀k₆r₂k₄ | d(MOP)r₆f₁₀k₆r₂k₄ |
| r(MP)f₆r₁₀k₆r₂k₄ | f(MOP)r₆f₁₀k₆r₂k₄ |
| r(MOP)r₁₆k₆r₂k₄ | r(MP)r₆f₁₀k₆r₂k₄ |
| d(MOP)r₁₆k₆r₂k₄ | d(MOP)r₆f₁₀k₆r₂k₄ |
| f(MOP)r₁₆k₆r₂k₄ | f(MOP)r₆f₁₀k₆r₂k₄ |
| r(MP)r₁₆k₆r₂k₄ | r(MOP)f₆r₁₀l₆r₂k₄ |
| d(MP)r₁₆k₆r₂k₄ | d(MOP)f₆r₁₀l₆r₂k₄ |
| f(MP)r₁₆k₆r₂k₄ | f(MOP)f₆r₁₀l₆r₂k₄ |
| r(MOP)r₆f₁₀k₆r₂k₄ | r(MOP)f₆r₁₀l₆r₂k₄ |
| d(MP)f₆r₁₀k₆r₂l₄ | d(MOP)f₆r₁₀f₆r₂l₄ |
| f(MP)f₆r₁₀k₆r₂l₄ | f(MOP)f₆r₁₀f₆r₂l₄ |
| r(MOP)r₁₆k₆r₂l₄ | f₇r₆kr₃kr₃kr₃k(G-Clamp)k₂ |
| d(MOP)r₁₆k₆r₂l₄ | mf₆r₆kr₃kr₃kr₃k(G-Clamp)rk₂ |
| f(MOP)r₁₆k₆r₂l₄ | mf₆r₁₀k₆r₂k(G-Clamp)k₂ |
| r(MP)r₁₆k₆r₂l₄ | mr₁₆k₆r₂k (G-Clam)k₂ |
| d(MP)r₁₆k₆r₂l₄ | mr₆f₁₀k₆r₂k (G-Clamp)k₂ |
| f(MP)r₁₆k₆r₂l₄ | mf₆r₁₀f₆r₂k(G-Clamp)k₂ |
| r(MOP)r₆f₁₀k₆r₂l₄ | mf₆r₁₀l₆r₂k(G-Clamp)k₂ |
| r(MOP)r₆f₁₀k₆r₂l₄ | mr₆f₁₀l₆r₂k(G-Clamp)k₂ |
| r(MOP)r₆f₁₀k₆r₂l₄ | mf₆r₁₀k₆r₂l(G-Clamp)l₂ |
| r(MOP)r₆f₁₀k₆r₂l₄ | mr₁₆k₆r₂l(G-Clamp)l₂ |
| r(MOP)r₆f₁₀k₆r₂l₄ | mr₆f₁₀k₆r₂l (G-Clamp)l₂ |
| r(MOP)r₆f₁₀k₆r₂l₄ | mf₆r₁₀f₆r₂l(G-Clamp)l₂ |
| r(MOP)f₆r₁₀f₆r₂l₄ | f₇r₆kr₃k(5-propyne)r₃kr₃krk₂ |
| d(MOP)f₆r₁₀f₆r₂l₄ | mf₆r₆kr₃k(5-Propyne)r₃kr₃krk₂ |
| f(MOP)f₆r₁₀f₆r₂l₄ | r(MOP)f₆r₁₀f₆r₂l₄ |

Table A Legend: “m” indicates a 2′-O-methyl modified nucleoside, “f” indicates a 2′-F modified nucleoside, “r” indicates an unmodified 2′-hydroxy sugar containing nucleoside, “d” indicates an unmodified 2′-deoxy sugar containing nucleoside, “e” indicates a 2′-MOE modified nucleoside, “k” indicates a cEt bicyclic sugar containing nucleoside, and “l” indicates an LNA bicyclic sugar containing nucleoside. The modifications listed in parentheses are optional modified nucleobases or optional modified internucleoside linkages: “(G-Clamp)” indicates a G-Clamp modified nucleobase that is part of the nucleoside represented by the letter immediately preceding it. “(5-Propyne)” indicates a 5′-propynyl modified nucleobase that is part of the nucleoside represented by the letter immediately preceding it. “(MOP)” indicates a methoxypropyl modified internucleoside linkage, “(MP)” indicates a methylphosphonate internucleoside linkage, and “(MMI)” indicates an MMI N-methyl internucleoside linkage. In certain embodiments, crRNAs having a motif with a parenthetical modification listed in the table above include the indicated parenthetical modification. In certain embodiments, the parenthetical modification of crRNAs having a motif with a parenthetical modification listed in the table above is replaced with a different modified or unmodified nucleobase or internucleoside linkage. The number subscripts in the table above indicate the number of contiguous nucleosides that comprise the identified modification. The lack of a number subscript indicates one nucleoside. The motifs listed in the table above may be used with any crRNA nucleobase sequence and with any internucleoside linkage motif. In certain embodiments, all of the nucleobases are unmodified. In certain embodiments, at least one nucleobase is a 5-methylcytosine modified nucleobase. In certain embodiments, the internucleoside linkages are all selected independently from among phosphate and phosphorothioate. In certain embodiments, one or more internucleoside linkages is a neutral internucleoside linkage.

D. mRNA

In certain embodiments, the cell surface receptor upregulating agent is mRNA encoding the cell surface receptor. In certain embodiments, the mRNA is modified. In certain embodiments, the mRNA encodes ASGPR or ASGPR1.

### Certain Cell Surface Receptors

Certain embodiments are directed to a method of targeting a non-native cell ectopically expressing a cell surface receptor. In certain embodiments, a method comprises contacting the non-native cell with a vector comprising a nucleic acid encoding the cell surface receptor.

Certain embodiments are directed to a method of delivering a compound to a subject ectopically expressing a cell surface receptor in non-native cells. In certain embodiments, a method comprises administering to the subject a vector comprising a nucleic acid encoding the cell surface receptor, thereby ectopically expressing the receptor in non-native cells in the subject.

In certain embodiments, the non-native cell does not endogenously express the receptor or endogenously expresses the receptor at low levels relative to a native cell that endogenously expresses the receptor.

In any of the aforementioned embodiments, the cell surface receptor can be ASGPR or ASGPR1. In any of the aforementioned embodiments, the cell surface receptor can be a G protein-coupled receptor (GPCR) including, but not limited to, the rhodopsin family of receptors, secretin family of receptors, glutamate family of receptors, adhesion family of receptors, and frizzled family of receptors. In any of the aforementioned embodiments, the cell surface receptor can be a glucagon family receptor including, but not limited to glucagon receptor, glucagon-like peptide 1 receptor, glucagon-like peptide 2 receptor, and gastric inhibitory polypeptide receptor. In any of the aforementioned embodiments, the cell surface receptor can be a folate receptor. In any of the aforementioned embodiments, the cell surface receptor can be a sigma receptor. In any of the aforementioned embodiments, the cell surface receptor can be an integrin receptor. In any of the aforementioned embodiments, the cell surface receptor can be a cannabinoid receptor. In any of the aforementioned embodiments, the cell surface receptor can be a gastrin-releasing peptide receptor.

In certain embodiments, a method of targeting a non-native cell ectopically expressing a cell surface receptor comprises contacting the non-native cell ectopically expressing the receptor with a compound comprising a modified oligonucleotide and a conjugate group, wherein the conjugate group binds to the receptor. In certain embodiments, the cell surface receptor is ASGPR or ASGPR1 and the conjugate group comprises GalNAc. In certain embodiments, the non-native cell is a non-liver cell, such as a brain, eye, muscle, heart, skin, kidney, lung, pancreas, intestinal, fat, spleen, bone, testes, ovary, pituitary, immune, or bladder cell.

In certain embodiments, a method of delivering a compound to a subject ectopically expressing a cell surface receptor in non-native cells comprises administering to the subject a compound comprising a modified oligonucleotide and a conjugate group, wherein the conjugate group binds to the receptor, thereby delivering the compound to non-native cells ectopically expressing the receptor in the subject. In certain embodiments, the cell surface receptor is ASGPR or ASGPR1 and the conjugate group comprises GalNAc. In certain embodiments, the non-native cell is a non-liver cell, such as a brain, eye, muscle, heart, skin, kidney, lung, pancreas, intestinal, fat, spleen, bone, testes, ovary, pituitary, immune, or bladder cell.

In any of the aforementioned embodiments, the conjugate group can be any ligand known in the art to bind to a receptor. In certain embodiments, the cell surface receptor is ASGPR or ASGPR1 and the conjugate group comprises GalNAc. In certain embodiments, the cell surface receptor is glucagon receptor and the conjugate group comprises glucagon. In certain embodiments, the cell surface receptor is glucagon-like peptide 1 receptor and the conjugate group comprises glucagon-like peptide 1. In certain embodiments, the cell surface receptor is glucagon-like peptide 2 receptor and the conjugate group comprises glucagon-like peptide 2. In certain embodiments, the cell surface receptor is a folate receptor and the conjugate group comprises folate. In certain embodiments, the cell surface receptor is a sigma receptor and the conjugate group comprises anisamide. In certain embodiments, the cell surface receptor is an integrin receptor and the conjugate group comprises the RGD tripeptide. In certain embodiments, the cell surface receptor is a cannabinoid receptor and the conjugate group comprises anandamide. In certain embodiments, the cell surface receptor is a gastrin-releasing peptide receptor and the conjugate group comprises bombesin.

It is understood in the art which cells are considered non-native with respect to expression of a given cell surface receptor. In certain embodiments, a non-liver cell is a non-native cell with respect to ASGPR or ASGPR1 expression. It has been reported that a hepatocyte expresses approximately 500,000 copies of ASGPR. Prakash T P et al., 2014 Nucleic Acids Res. 42(13):8796-807, which is incorporated by reference in its entirety herein.

In certain embodiments, a non-native cell is any kind of cell (e.g. tissue type or sub-tissue type) that endogenously expresses fewer copies of a given cell surface receptor than the kind of cell that endogenously expresses the most copies of the same given cell surface receptor. For example, but not by limitation, in certain embodiments any non-liver cell is a non-native cell with respect to ASGPR expression, whereas a liver cell endogenously expresses the most copies of ASGPR.

In certain embodiments, a non-native cell endogenously expresses fewer than 1,000,000 copies of a given cell surface receptor. In certain embodiments, a non-native cell endogenously expresses fewer than 900,000 copies of a given cell surface receptor. In certain embodiments, a non-native cell endogenously expresses fewer than 800,000 copies of a given cell surface receptor. In certain embodiments, a non-native cell endogenously expresses fewer than 700,000 copies of a given cell surface receptor. In certain embodiments, a non-native cell endogenously expresses fewer than 600,000 copies of a given cell surface receptor. In certain embodiments, a non-native cell endogenously expresses fewer than 500,000 copies of a given cell surface receptor. In certain embodiments, a non-native cell endogenously expresses fewer than 400,000 copies of a given cell surface receptor. In certain embodiments, a non-native cell endogenously expresses fewer than 300,000 copies of a given cell surface receptor. In certain embodiments, anon-native cell endogenously expresses fewer than 200,000 copies of a given cell surface receptor. In certain embodiments, a non-native cell endogenously expresses fewer than 100,000 copies of a given cell surface receptor. In certain embodiments, a non-native cell endogenously expresses fewer than 90,000 copies of a given cell surface receptor. In certain embodiments, a non-native cell endogenously expresses fewer than 80,000 copies of a given cell surface receptor. In certain embodiments, a non-native cell endogenously expresses fewer than 70,000 copies of a given cell surface receptor. In certain embodiments, a non-native cell endogenously expresses fewer than 60,000 copies of a given cell surface receptor. In certain embodiments, a non-native cell endogenously expresses fewer than 50,000 copies of a given cell surface receptor. In certain embodiments, a non-native cell endogenously expresses fewer than 40,000 copies of a given cell surface receptor. In certain embodiments, a non-native cell endogenously expresses fewer than 30,000 copies of a given cell surface receptor. In certain embodiments, a non-native cell endogenously expresses fewer than 20,000 copies of a given cell surface receptor. In certain embodiments, a non-native cell endogenously expresses fewer than 10,000 copies of a given cell surface receptor. In certain embodiments, a non-native cell endogenously expresses fewer than 9,000 copies of a given cell surface receptor. In certain embodiments, a non-native cell endogenously expresses fewer than 8,000 copies of a given cell surface receptor. In certain embodiments, a non-native cell endogenously expresses fewer than 7,000 copies of a given cell surface receptor. In certain embodiments, a non-native cell endogenously expresses fewer than 6,000 copies of a given cell surface receptor. In certain embodiments, a non-native cell endogenously expresses fewer than 5,000 copies of a given cell surface receptor. In certain embodiments, a non-native cell endogenously expresses fewer than 4,000 copies of a given cell surface receptor. In certain embodiments, a non-native cell endogenously expresses fewer than 3,000 copies of a given cell surface receptor. In certain embodiments, a non-native cell endogenously expresses fewer than 2,000 copies of a given cell surface receptor. In certain embodiments, a non-native cell endogenously expresses fewer than 1,000 copies of a given cell surface receptor. In certain embodiments, a non-native cell endogenously expresses fewer than 900 copies of a given cell surface receptor. In certain embodiments, a non-native cell endogenously expresses fewer than 800 copies of a given cell surface receptor. In certain embodiments, a non-native cell endogenously expresses fewer than 700 copies of a given cell surface receptor. In certain embodiments, a non-native cell endogenously expresses fewer than 600 copies of a given cell surface receptor. In certain embodiments, a non-native cell endogenously expresses fewer than 500 copies of a given cell surface receptor. In certain embodiments, a non-native cell endogenously expresses fewer than 400 copies of a given cell surface receptor. In certain embodiments, a non-native cell endogenously expresses fewer than 300 copies of a given cell surface receptor. In certain embodiments, a non-native cell endogenously expresses fewer than 200 copies of a given cell surface receptor. In certain embodiments, a non-native cell endogenously expresses fewer than 100 copies of a given cell surface receptor.

### Certain Conjugated Compounds

In certain embodiments, the compounds described herein comprise or consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides.

In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

In certain embodiments, the oligonucleotide is modified. In certain embodiments, the oligonucleotide of a compound has a nucleobase sequence that is complementary to a target nucleic acid. In certain embodiments, oligonucleotides are complementary to a messenger RNA (mRNA). In certain embodiments, oligonucleotides are complementary to a sense transcript. In certain embodiments, oligonucleotides are complementary to an antisense transcript. In certain embodiments, oligonucleotides are complementary to a long non-coding RNA (lncRNA).

Examples of terminal groups include but are not limited to conjugate groups, capping groups, phosphate moieties, protecting groups, modified or unmodified nucleosides, and two or more nucleosides that are independently modified or unmodified.

### Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In certain embodiments, conjugate groups impart a new property on the attached oligonucleotide, e.g., fluorophores or reporter groups that enable detection of the oligonucleotide.

Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), athiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic, a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, i, 923-937), a tocopherol group (Nishina et al., Molecular Therapy Nucleic Acids, 2015, 4, e220; doi:10.1038/mtna.2014.72 and Nishina et al., Molecular Therapy, 2008, 16, 734-740), or a GalNAc cluster (e.g., WO2014/179620).

1. Conjugate Moieties

Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

2. Conjugate Linkers

Conjugate moieties are attached to oligonucleotides through conjugate linkers. In certain compounds, a conjugate group is a single chemical bond (i.e. conjugate moiety is attached to an oligonucleotide via a conjugate linker through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C1-C10 alkyl, substituted or unsubstituted C2-C10 alkenyl or substituted or unsubstituted C2-C10 alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the compound after it reaches a target tissue. Accordingly, linker-nucleosides are typically linked to one another and to the remainder of the compound through cleavable bonds. In certain embodiments, such cleavable bonds are phosphodiester bonds.

Herein, linker-nucleosides are not considered to be part of the oligonucleotide. Accordingly, in embodiments in which a compound comprises an oligonucleotide consisting of a specified number or range of linked nucleosides and/or a specified percent complementarity to a reference nucleic acid and the compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, a compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such a compound is more than 30. Alternatively, a compound may comprise a modified oligonucleotide consisting of 8-30 nucleosides and no conjugate group. The total number of contiguous linked nucleosides in such a compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances compounds comprising a particular conjugate moiety are better taken up by a particular cell type, but once the compound has been taken up, it is desirable that the conjugate group be cleaved to release the unconjugated or parent oligonucleotide. Thus, certain conjugate may comprise one or more cleavable moieties, typically within the conjugate linker. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, one or more linker-nucleosides are linked to one another and/or to the remainder of the compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

3. Certain Cell-Targeting Conjugate Moieties

In certain embodiments, a conjugate group comprises a cell-targeting conjugate moiety. In certain embodiments, a conjugate group has the general formula:

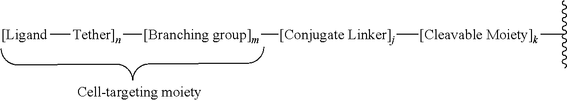

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1.

In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, the cell-targeting moiety comprises a branching group comprising one or more groups selected from alkyl, amino, oxo, amide, disulfide, polyethylene glycol, ether, thioether and hydroxylamino groups. In certain embodiments, the branching group comprises a branched aliphatic group comprising groups selected from alkyl, amino, oxo, amide, disulfide, polyethylene glycol, ether, thioether and hydroxylamino groups. In certain such embodiments, the branched aliphatic group comprises groups selected from alkyl, amino, oxo, amide and ether groups. In certain such embodiments, the branched aliphatic group comprises groups selected from alkyl, amino and ether groups. In certain such embodiments, the branched aliphatic group comprises groups selected from alkyl and ether groups. In certain embodiments, the branching group comprises a mono or polycyclic ring system.

In certain embodiments, each tether of a cell-targeting moiety comprises one or more groups selected from alkyl, substituted alkyl, ether, thioether, disulfide, amino, oxo, amide, phosphodiester, and polyethylene glycol, in any combination. In certain embodiments, each tether is a linear aliphatic group comprising one or more groups selected from alkyl, ether, thioether, disulfide, amino, oxo, amide, and polyethylene glycol, in any combination. In certain embodiments, each tether is a linear aliphatic group comprising one or more groups selected from alkyl, phosphodiester, ether, amino, oxo, and amide, in any combination. In certain embodiments, each tether is a linear aliphatic group comprising one or more groups selected from alkyl, ether, amino, oxo, and amid, in any combination. In certain embodiments, each tether is a linear aliphatic group comprising one or more groups selected from alkyl, amino, and oxo, in any combination. In certain embodiments, each tether is a linear aliphatic group comprising one or more groups selected from alkyl and oxo, in any combination. In certain embodiments, each tether is a linear aliphatic group comprising one or more groups selected from alkyl and phosphodiester, in any combination. In certain embodiments, each tether comprises at least one phosphorus linking group or neutral linking group. In certain embodiments, each tether comprises a chain from about 6 to about 20 atoms in length. In certain embodiments, each tether comprises a chain from about 10 to about 18 atoms in length. In certain embodiments, each tether comprises about 10 atoms in chain length.

In certain embodiments, each ligand of a cell-targeting moiety has an affinity for at least one type of receptor on a target cell. In certain embodiments, each ligand has an affinity for at least one type of receptor on the surface of a mammalian liver cell. In certain embodiments, each ligand has an affinity for the hepatic asialoglycoprotein receptor (ASGP-R). In certain embodiments, each ligand is a carbohydrate. In certain embodiments, each ligand is, independently selected from galactose, N-acetyl galactoseamine (GalNAc), mannose, glucose, glucoseamine and fucose. In certain embodiments, each ligand is N-acetyl galactoseamine (GalNAc). In certain embodiments, the cell-targeting moiety comprises 3 GalNAc ligands. In certain embodiments, the cell-targeting moiety comprises 2 GalNAc ligands. In certain embodiments, the cell-targeting moiety comprises 1 GalNAc ligand.

In certain embodiments, each ligand of a cell-targeting moiety is a carbohydrate, carbohydrate derivative, modified carbohydrate, polysaccharide, modified polysaccharide, or polysaccharide derivative. In certain such embodiments, the conjugate group comprises a carbohydrate cluster (see, e.g., Maier et al., “Synthesis of Antisense Oligonucleotides Conjugated to a Multivalent Carbohydrate Cluster for Cellular Targeting,” Bioconjugate Chemistry, 2003, 14, 18-29, or Rensen et al., “Design and Synthesis of Novel N-Acetylgalactosamine-Terminated Glycolipids for Targeting of Lipoproteins to the Hepatic Asiaglycoprotein Receptor,” J. Med. Chem. 2004, 47, 5798-5808, which are incorporated herein by reference in their entirety). In certain such embodiments, each ligand is an amino sugar or a thio sugar. For example, amino sugars may be selected from any number of compounds known in the art, such as sialic acid, α-D-galactosamine, β-muramic acid, 2-deoxy-2-methylamino-L-glucopyranose, 4,6-dideoxy-4-formamido-2,3-di-O-methyl-D-mannopyranose, 2-deoxy-2-sulfoamino-D-glucopyranose and N-sulfo-D-glucosamine, and N-glycoloyl-α-neuraminic acid. For example, thio sugars may be selected from 5-Thio-β-D-glucopyranose, methyl 2,3,4-tri-O-acetyl-1-thio-6-O-trityl-α-D-glucopyranoside, 4-thio-β-D-galactopyranose, and ethyl 3,4,6,7-tetra-O-acetyl-2-deoxy-1,5-dithio-α-D-gluco-heptopyranoside.

In certain embodiments, conjugate groups comprise a cell-targeting moiety having the formula:

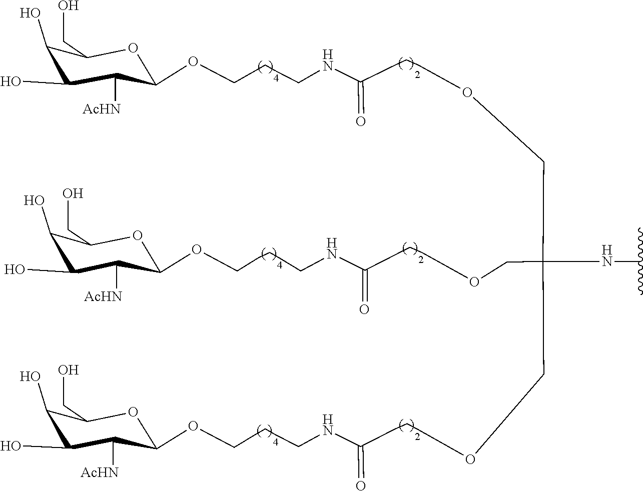

In certain embodiments, conjugate groups comprise a cell-targeting moiety having the formula:

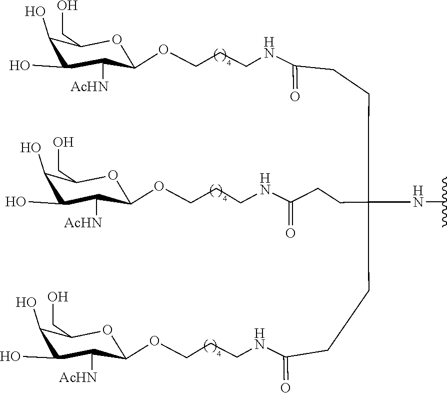

In certain embodiments, conjugate groups comprise a cell-targeting moiety having the formula:

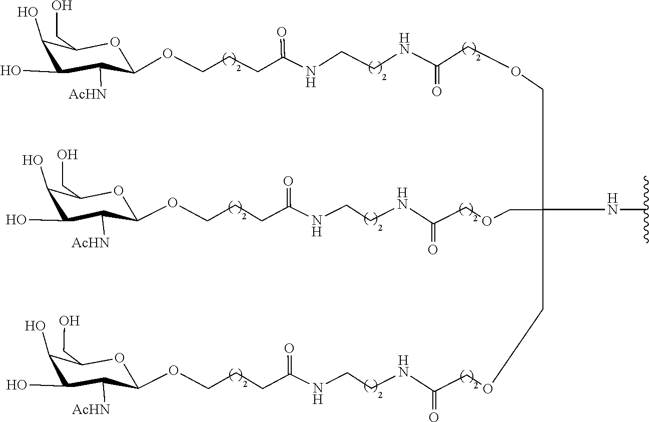

In certain embodiments, compounds described herein comprise a conjugate group described herein as “LICA-1”. LICA-1 is shown below without the optional cleavable moiety at the end of the conjugate linker:

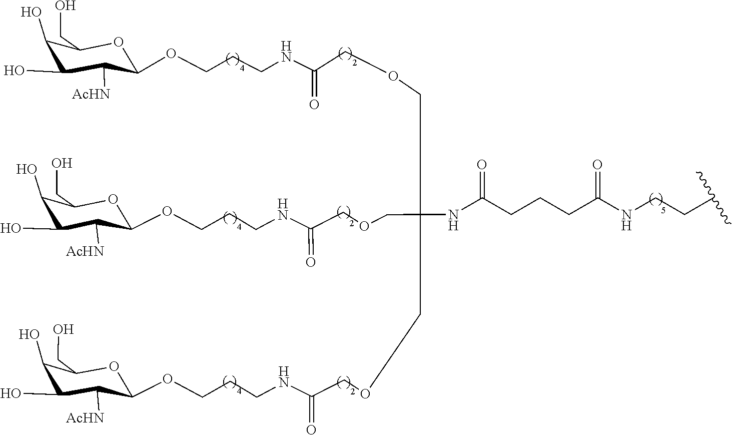

In certain embodiments, compounds described herein comprise LICA-1 and a cleavable moiety within the conjugate linker have the formula:

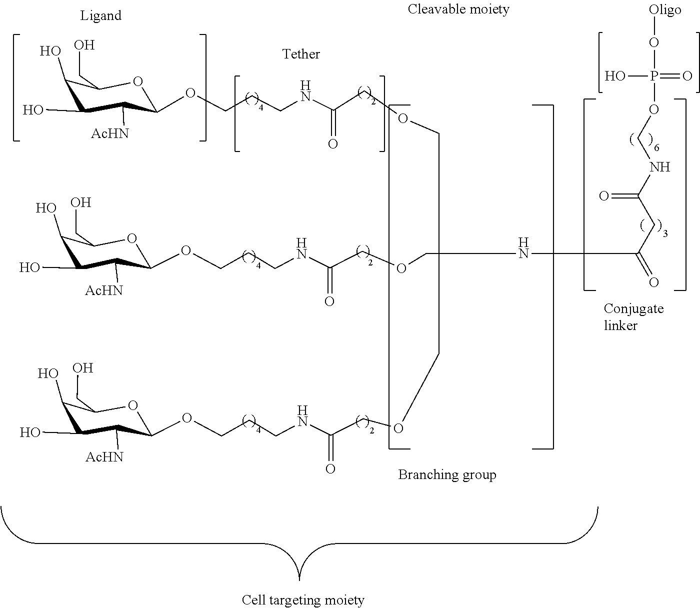

wherein oligo is an oligonucleotide.

Representative publications that teach the preparation of certain of the above noted conjugate groups and compounds comprising conjugate groups, tethers, conjugate linkers, branching groups, ligands, cleavable moieties as well as other modifications include without limitation, U.S. Pat. Nos. 5,994,517, 6,300,319, 6,660,720, 6,906,182, 7,262,177, 7,491,805, 8,106,022, 7,723,509, US 2006/0148740, US 2011/0123520, WO 2013/033230 and WO 2012/037254, Biessen et al., J. Med. Chem. 1995, 38, 1846-1852, Lee et al., Bioorganic & Medicinal Chemistry 2011, 19, 2494-2500, Rensen et al., J. Biol. Chem. 2001, 276, 37577-37584, Rensen et al., J. Med. Chem. 2004, 47, 5798-5808, Sliedregt et al., J. Med. Chem. 1999, 42, 609-618, and Valentijn et al., Tetrahedron, 1997, 53, 759-770, each of which is incorporated by reference herein in its entirety.

In certain embodiments, compounds described herein comprise modified oligonucleotides comprising a gapmer or fully modified motif and a conjugate group comprising at least one, two, or three GalNAc ligands. In certain embodiments compounds described herein comprise a conjugate group found in any of the following references: Lee, Carbohydr Res, 1978, 67, 509-514; Connolly et al., J Biol Chem, 1982, 257, 939-945; Pavia et al., Int J Pep Protein Res, 1983, 22, 539-548; Lee et al., Biochem, 1984, 23, 4255-4261; Lee et al., Glycoconjugate J, 1987, 4, 317-328; Toyokuni et al., Tetrahedron Lett, 1990, 31, 2673-2676; Biessen et al., J Med Chem, 1995, 38, 1538-1546; Valentijn et al., Tetrahedron, 1997, 53, 759-770; Kim et al., Tetrahedron Lett, 1997, 38, 3487-3490; Lee et al., Bioconjug Chem, 1997, 8, 762-765; Kato et al., Glycobiol, 2001, 11, 821-829; Rensen et al., J Biol Chem, 2001, 276, 37577-37584; Lee et al., Methods Enzymol, 2003, 362, 38-43; Westerlind et al., Glycoconj J, 2004, 21, 227-241; Lee et al., Bioorg Med Chem Lett, 2006, 16(19), 5132-5135; Maierhofer et al., Bioorg Med Chem, 2007, 15, 7661-7676; Khorev et al., Bioorg Med Chem, 2008, 16, 5216-5231; Lee et al., Bioorg Med Chem, 2011, 19, 2494-2500; Komilova et al., Analyt Biochem, 2012, 425, 43-46; Pujol et al., Angew Chemie Int Ed Engl, 2012, 51, 7445-7448; Biessen et al., J Med Chem, 1995, 38, 1846-1852; Sliedregt et al., J Med Chem, 1999, 42, 609-618; Rensen et al., J Med Chem, 2004, 47, 5798-5808; Rensen et al., Arterioscler Thromb Vase Biol, 2006, 26, 169-175; van Rossenberg et al., Gene Ther, 2004, 11, 457-464; Sato et al., J Am Chem Soc, 2004, 126, 14013-14022; Lee et al., J Org Chem, 2012, 77, 7564-7571; Biessen et al., FASEB J, 2000, 14, 1784-1792; Rajur et al., Bioconjug Chem, 1997, 8, 935-940; Duff et al., Methods Enzymol, 2000, 313, 297-321; Maier et al., Bioconjug Chem, 2003, 14, 18-29; Jayaprakash et al., Org Lett, 2010, 12, 5410-5413; Manoharan, Antisense Nucleic Acid Drug Dev, 2002, 12, 103-128; Merwin et al., Bioconjug Chem, 1994, 5, 612-620; Tomiya et al., Bioorg Med Chem, 2013, 21, 5275-5281; International applications WO1998/013381; WO2011/038356; WO1997/046098; WO2008/098788; WO2004/101619; WO2012/037254; WO2011/120053; WO2011/100131; WO2011/163121; WO2012/177947; WO2013/033230; WO2013/075035; WO2012/083185; WO2012/083046; WO2009/082607; WO2009/134487; WO2010/144740; WO2010/148013; WO1997/020563; WO2010/088537; WO2002/043771; WO2010/129709; WO2012/068187; WO2009/126933; WO2004/024757; WO2010/054406; WO2012/089352; WO2012/089602; WO2013/166121; WO2013/165816; U.S. Pat. Nos. 4,751,219; 8,552,163; 6,908,903; 7,262,177; 5,994,517; 6,300,319; 8,106,022; 7,491,805; 7,491,805; 7,582,744; 8,137,695; 6,383,812; 6,525,031; 6,660,720; 7,723,509; 8,541,548; 8,344,125; 8,313,772; 8,349,308; 8,450,467; 8,501,930; 8,158,601; 7,262,177; 6,906,182; 6,620,916; 8,435,491; 8,404,862; 7,851,615; Published U.S. Patent Application Publications US2011/0097264; US2011/0097265; US2013/0004427; US2005/0164235; US2006/0148740; US2008/0281044; US2010/0240730; US2003/0119724; US2006/0183886; US2008/0206869; US2011/0269814; US2009/0286973; US2011/0207799; US2012/0136042; US2012/0165393; US2008/0281041; US2009/0203135; US2012/0035115; US2012/0095075; US2012/0101148; US2012/0128760; US2012/0157509; US2012/0230938; US2013/0109817; US2013/0121954; US2013/0178512; US2013/0236968; US2011/0123520; US2003/0077829; US2008/0108801; and US2009/0203132; each of which is incorporated by reference in its entirety.

### Certain Compounds

In certain embodiments, compounds described herein are antisense compounds. In certain embodiments, the antisense compound comprises or consists of an oligomeric compound. In certain embodiments, the oligomeric compound comprises a modified oligonucleotide. In certain embodiments, the modified oligonucleotide has a nucleobase sequence complementary to that of a target nucleic acid.

In certain embodiments, a compound described herein comprises or consists of a modified oligonucleotide. In certain embodiments, the modified oligonucleotide has a nucleobase sequence complementary to that of a target nucleic acid.

In certain embodiments, a compound or antisense compound is single-stranded. Such a single-stranded compound or antisense compound comprises or consists of an oligomeric compound. In certain embodiments, such an oligomeric compound comprises or consists of an oligonucleotide. In certain embodiments, the oligonucleotide is an antisense oligonucleotide. In certain embodiments, the oligonucleotide is modified. In certain embodiments, the oligonucleotide of a single-stranded antisense compound or oligomeric compound comprises a self-complementary nucleobase sequence.

In certain embodiments, compounds are double-stranded. Such double-stranded compounds comprise a first modified oligonucleotide having a region complementary to a target nucleic acid and a second modified oligonucleotide having a region complementary to the first modified oligonucleotide. In certain embodiments, the modified oligonucleotide is an RNA oligonucleotide. In such embodiments, the thymine nucleobase in the modified oligonucleotide is replaced by a uracil nucleobase. In certain embodiments, compound comprises a conjugate group. In certain embodiments, each modified oligonucleotide is 12-30 linked nucleosides in length.

In certain embodiments, compounds are double-stranded. Such double-stranded compounds comprise a first oligomeric compound having a region complementary to a target nucleic acid and a second oligomeric compound having a region complementary to the first oligomeric compound. The first oligomeric compound of such double stranded compounds typically comprises or consists of a modified oligonucleotide. The oligonucleotide of the second oligomeric compound of such double-stranded compound may be modified or unmodified. The oligomeric compounds of double-stranded compounds may include non-complementary overhanging nucleosides.

Examples of single-stranded and double-stranded compounds include but are not limited to oligonucleotides, siRNAs, microRNA targeting oligonucleotides, and single-stranded RNAi compounds, such as small hairpin RNAs (shRNAs), single-stranded siRNAs (ssRNAs), and microRNA mimics.

In certain embodiments, a compound described herein has a nucleobase sequence that, when written in the 5′ to 3′ direction, comprises the reverse complement of the target segment of a target nucleic acid to which it is targeted.

In certain embodiments, a compound described herein comprises an oligonucleotide 10 to 30 linked subunits in length. In certain embodiments, compound described herein comprises an oligonucleotide is 12 to 30 linked subunits in length. In certain embodiments, compound described herein comprises an oligonucleotide is 12 to 22 linked subunits in length. In certain embodiments, compound described herein comprises an oligonucleotide is 14 to 30 linked subunits in length. In certain embodiments, compound described herein comprises an oligonucleotide is 14 to 20 linked subunits in length. In certain embodiments, compound described herein comprises an oligonucleotide is 15 to 30 linked subunits in length. In certain embodiments, compound described herein comprises an oligonucleotide is 15 to 20 linked subunits in length. In certain embodiments, compound described herein comprises an oligonucleotide is 16 to 30 linked subunits in length. In certain embodiments, compound described herein comprises an oligonucleotide is 16 to 20 linked subunits in length. In certain embodiments, compound described herein comprises an oligonucleotide is 17 to 30 linked subunits in length. In certain embodiments, compound described herein comprises an oligonucleotide is 17 to 20 linked subunits in length. In certain embodiments, compound described herein comprises an oligonucleotide is 18 to 30 linked subunits in length. In certain embodiments, compound described herein comprises an oligonucleotide is 18 to 21 linked subunits in length. In certain embodiments, compound described herein comprises an oligonucleotide is 18 to 20 linked subunits in length. In certain embodiments, compound described herein comprises an oligonucleotide is 20 to 30 linked subunits in length. In other words, such oligonucleotides are from 12 to 30 linked subunits, 14 to 30 linked subunits, 14 to 20 subunits, 15 to 30 subunits, 15 to 20 subunits, 16 to 30 subunits, 16 to 20 subunits, 17 to 30 subunits, 17 to 20 subunits, 18 to 30 subunits, 18 to 20 subunits, 18 to 21 subunits, 20 to 30 subunits, or 12 to 22 linked subunits, respectively. In certain embodiments, a compound described herein comprises an oligonucleotide 14 linked subunits in length. In certain embodiments, a compound described herein comprises an oligonucleotide 16 linked subunits in length. In certain embodiments, a compound described herein comprises an oligonucleotide 17 linked subunits in length. In certain embodiments, compound described herein comprises an oligonucleotide 18 linked subunits in length. In certain embodiments, a compound described herein comprises an oligonucleotide 19 linked subunits in length. In certain embodiments, a compound described herein comprises an oligonucleotide 20 linked subunits in length. In other embodiments, a compound described herein comprises an oligonucleotide 8 to 80, 12 to 50, 13 to 30, 13 to 50, 14 to 30, 14 to 50, 15 to 30, 15 to 50, 16 to 30, 16 to 50, 17 to 30, 17 to 50, 18 to 22, 18 to 24, 18 to 30, 18 to 50, 19 to 22, 19 to 30, 19 to 50, or 20 to 30 linked subunits. In certain such embodiments, the compound described herein comprises an oligonucleotide 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked subunits in length, or a range defined by any two of the above values. In some embodiments the linked subunits are nucleotides, nucleosides, or nucleobases.

In certain embodiments, compounds may be shortened or truncated. For example, a single subunit may be deleted from the 5′ end (5′ truncation), or alternatively from the 3′ end (3′ truncation). A shortened or truncated compound targeted to a target gene nucleic acid may have two subunits deleted from the 5′ end, or alternatively may have two subunits deleted from the 3′ end, of the compound. Alternatively, the deleted nucleosides may be dispersed throughout the compound.

When a single additional subunit is present in a lengthened compound, the additional subunit may be located at the 5′ or 3′ end of the compound. When two or more additional subunits are present, the added subunits may be adjacent to each other, for example, in a compound having two subunits added to the 5′ end (5′ addition), or alternatively to the 3′ end (3′ addition), of the compound. Alternatively, the added subunits may be dispersed throughout the compound.

It is possible to increase or decrease the length of a compound, such as an oligonucleotide, and/or introduce mismatch bases without eliminating activity (Woolf et al. (Proc. Natl. Acad. Sci. USA 89:7305-7309, 1992; Gautschi et al. J Natd. Cancer Inst. 93:463-471, March 2001; Maher and Dolnick Nuc. Acid. Res. 16:3341-3358, 1988). However, seemingly small changes in oligonucleotide sequence, chemistry and motif can make large differences in one or more of the many properties required for clinical development (Seth et al. J. Med. Chem. 2009, 52, 10; Egli et al. J. Am. Chem. Soc. 2011, 133, 16642).

In certain embodiments, compounds described herein are interfering RNA compounds (RNAi), which include double-stranded RNA compounds (also referred to as short-interfering RNA or siRNA) and single-stranded RNAi compounds (or ssRNA). Such compounds work at least in part through the RISC pathway to degrade and/or sequester a target nucleic acid (thus, include microRNA/microRNA-mimic compounds). As used herein, the term siRNA is meant to be equivalent to other terms used to describe nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically modified siRNA, post-transcriptional gene silencing RNA (ptgsRNA), and others. In addition, as used herein, the term RNAi is meant to be equivalent to other terms used to describe sequence specific RNA interference, such as post transcriptional gene silencing, translational inhibition, or epigenetics.

In certain embodiments, a double-stranded compound comprises a first strand comprising the nucleobase sequence complementary to a target region of a target gene nucleic acid and a second strand. In certain embodiments, the double-stranded compound comprises ribonucleotides in which the first strand has uracil (U) in place of thymine (T) and is complementary to a target region. In certain embodiments, a double-stranded compound comprises (i) a first strand comprising a nucleobase sequence complementary to a target region of a target gene nucleic acid, and (ii) a second strand. In certain embodiments, the double-stranded compound comprises one or more modified nucleotides in which the 2′ position in the sugar contains a halogen (such as fluorine group; 2′-F) or contains an alkoxy group (such as a methoxy group; 2′-OMe). In certain embodiments, the double-stranded compound comprises at least one 2′-F sugar modification and at least one 2′-OMe sugar modification. In certain embodiments, the at least one 2′-F sugar modification and at least one 2′-OMe sugar modification are arranged in an alternating pattern for at least 2, 3, 4, 5, 6, 7, 8, 9, 10, 11, 12, 13, 14, 15, 16, 17, 18, 19, or 20 contiguous nucleobases along a strand of the dsRNA compound. In certain embodiments, the double-stranded compound comprises one or more linkages between adjacent nucleotides other than a naturally-occurring phosphodiester linkage. Examples of such linkages include phosphoramide, phosphorothioate, and phosphorodithioate linkages. The double-stranded compounds may also be chemically modified nucleic acid molecules as taught in U.S. Pat. No. 6,673,661. In other embodiments, the dsRNA contains one or two capped strands, as disclosed, for example, by WO 00/63364, filed Apr. 19, 2000. In certain embodiments, the first strand of the double-stranded compound is an siRNA guide strand and the second strand of the double-stranded compound is an siRNA passenger strand. In certain embodiments, the second strand of the double-stranded compound is complementary to the first strand. In certain embodiments, each strand of the double-stranded compound consists of 16, 17, 18, 19, 20, 21, 22, or 23 linked nucleosides.

In certain embodiments, a single-stranded compound described herein can comprise any of the oligonucleotide sequences targeted to a target gene. In certain embodiments, such a single-stranded compound is a single-stranded RNAi (ssRNAi) compound. In certain embodiments, a ssRNAi compound comprises the nucleobase sequence complementary to a target region of a target gene nucleic acid. In certain embodiments, the ssRNAi compound comprises ribonucleotides in which uracil (U) is in place of thymine (T). In certain embodiments, ssRNAi compound comprises a nucleobase sequence complementary to a target region of a target gene nucleic acid. In certain embodiments, a ssRNAi compound comprises one or more modified nucleotides in which the 2′ position in the sugar contains a halogen (such as fluorine group; 2′-F) or contains an alkoxy group (such as a methoxy group; 2′-OMe). In certain embodiments, a ssRNAi compound comprises at least one 2′-F sugar modification and at least one 2′-OMe sugar modification. In certain embodiments, the at least one 2′-F sugar modification and at least one 2′-OMe sugar modification are arranged in an alternating pattern for at least 2, 3, 4, 5, 6, 7, 8, 9, 10, 11, 12, 13, 14, 15, 16, 17, 18, 19, or 20 contiguous nucleobases along a strand of the ssRNAi compound. In certain embodiments, the ssRNAi compound comprises one or more linkages between adjacent nucleotides other than a naturally-occurring phosphodiester linkage. Examples of such linkages include phosphoramide, phosphorothioate, and phosphorodithioate linkages. The ssRNAi compounds may also be chemically modified nucleic acid molecules as taught in U.S. Pat. No. 6,673,661. In other embodiments, the ssRNAi contains a capped strand, as disclosed, for example, by WO 00/63364, filed Apr. 19, 2000. In certain embodiments, the ssRNAi compound consists of 16, 17, 18, 19, 20, 21, 22, or 23 linked nucleosides.

In certain embodiments, compounds described herein comprise modified oligonucleotides. Certain modified oligonucleotides have one or more asymmetric center and thus give rise to enantiomers, diastereomers, and other stereoisomeric configurations that may be defined, in terms of absolute stereochemistry, as (R) or (S), as a or B such as for sugar anomers, or as (D) or (L) such as for amino acids etc. Included in the modified oligonucleotides provided herein are all such possible isomers, including their racemic and optically pure forms, unless specified otherwise. Likewise, all cis- and trans-isomers and tautomeric forms are also included.

### Certain Mechanisms

In certain embodiments, compounds described herein comprise or consist of modified oligonucleotides. In certain embodiments, compounds described herein are antisense compounds. In certain embodiments, such antisense compounds comprise oligomeric compounds. In certain embodiments, compounds described herein are capable of hybridizing to a target nucleic acid, resulting in at least one antisense activity. In certain embodiments, compounds described herein selectively affect one or more target nucleic acid. Such selective compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in a significant undesired antisense activity.

In certain antisense activities, hybridization of a compound described herein to a target nucleic acid results in recruitment of a protein that cleaves the target nucleic acid. For example, certain compounds described herein result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, compounds described herein are sufficiently “DNA-like”to elicit RNase H activity. Further, in certain embodiments, one or more non-DNA-like nucleoside in the gap of a gapmer is tolerated.

In certain antisense activities, compounds described herein or a portion of the compound is loaded into an RNA-induced silencing complex (RISC), ultimately resulting in cleavage of the target nucleic acid. For example, certain compounds described herein result in cleavage of the target nucleic acid by Argonaute. Compounds that are loaded into RISC are RNAi compounds. RNAi compounds may be double-stranded (siRNA) or single-stranded (ssRNA).

In certain embodiments, hybridization of compounds described herein to a target nucleic acid does not result in recruitment of a protein that cleaves that target nucleic acid. In certain such embodiments, hybridization of the compound to the target nucleic acid results in alteration of splicing of the target nucleic acid. In certain embodiments, hybridization of the compound to a target nucleic acid results in inhibition of a binding interaction between the target nucleic acid and a protein or other nucleic acid. In certain such embodiments, hybridization of the compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein, and/or a phenotypic change in a cell or individual.

### Complementarity

An oligonucleotide is said to be complementary to another nucleic acid when the nucleobase sequence of such oligonucleotide or one or more regions thereof matches the nucleobase sequence of another oligonucleotide or nucleic acid or one or more regions thereof when the two nucleobase sequences are aligned in opposing directions. Nucleobase matches or complementary nucleobases, as described herein, are limited to adenine (A) and thymine (T), adenine (A) and uracil (U), cytosine (C) and guanine (G), and 5-methyl cytosine (mC) and guanine (G) unless otherwise specified. Complementary oligonucleotides and/or nucleic acids need not have nucleobase complementarity at each nucleoside and may include one or more nucleobase mismatches. An oligonucleotide is fully complementary or 100% complementary when such oligonucleotides have nucleobase matches at each nucleoside without any nucleobase mismatches.

In certain embodiments, compounds described herein comprise or consist of modified oligonucleotides. In certain embodiments, compounds described herein are antisense compounds. In certain embodiments, compounds comprise oligomeric compounds. Non-complementary nucleobases between a compound and a target gene nucleic acid may be tolerated provided that the compound remains able to specifically hybridize to a target nucleic acid. Moreover, a compound may hybridize over one or more segments of a target gene nucleic acid such that intervening or adjacent segments are not involved in the hybridization event (e.g., a loop structure, mismatch or hairpin structure).

In certain embodiments, the compounds provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a target gene nucleic acid, a target region, target segment, or specified portion thereof. Percent complementarity of a compound with a target nucleic acid can be determined using routine methods.

For example, a compound in which 18 of 20 nucleobases of the compound are complementary to a target region, and would therefore specifically hybridize, would represent 90 percent complementarity. In this example, the remaining non-complementary nucleobases may be clustered or interspersed with complementary nucleobases and need not be contiguous to each other or to complementary nucleobases. As such, a compound which is 18 nucleobases in length having four non-complementary nucleobases which are flanked by two regions of complete complementarity with the target nucleic acid would have 77.8% overall complementarity with the target nucleic acid and would thus fall within the scope of the present invention. Percent complementarity of a compound with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, compounds described herein, or specified portions thereof, are fully complementary (i.e. 100% complementary) to a target nucleic acid, or specified portion thereof. For example, a compound may be fully complementary to a target gene nucleic acid, or a target region, or a target segment or target sequence thereof. As used herein, “fully complementary” means each nucleobase of a compound is capable of precise base pairing with the corresponding nucleobases of a target nucleic acid. For example, a 20 nucleobase compound is fully complementary to a target sequence that is 400 nucleobases long, so long as there is a corresponding 20 nucleobase portion of the target nucleic acid that is fully complementary to the compound. Fully complementary can also be used in reference to a specified portion of the first and/or the second nucleic acid. For example, a 20 nucleobase portion of a 30 nucleobase compound can be “fully complementary” to a target sequence that is 400 nucleobases long. The 20 nucleobase portion of the 30 nucleobase compound is fully complementary to the target sequence if the target sequence has a corresponding 20 nucleobase portion wherein each nucleobase is complementary to the 20 nucleobase portion of the compound. At the same time, the entire 30 nucleobase compound may or may not be fully complementary to the target sequence, depending on whether the remaining 10 nucleobases of the compound are also complementary to the target sequence.

In certain embodiments, compounds described herein comprise one or more mismatched nucleobases relative to the target nucleic acid. In certain such embodiments, antisense activity against the target is reduced by such mismatch, but activity against a non-target is reduced by a greater amount. Thus, in certain such embodiments selectivity of the compound is improved. In certain embodiments, the mismatch is specifically positioned within an oligonucleotide having a gapmer motif. In certain such embodiments, the mismatch is at position 1, 2, 3, 4, 5, 6, 7, or 8 from the 5′-end of the gap region. In certain such embodiments, the mismatch is at position 9, 8, 7, 6, 5, 4, 3, 2, 1 from the 3′-end of the gap region. In certain such embodiments, the mismatch is at position 1, 2, 3, or 4 from the 5′-end of the wing region. In certain such embodiments, the mismatch is at position 4, 3, 2, or 1 from the 3′-end of the wing region. In certain embodiments, the mismatch is specifically positioned within an oligonucleotide not having a gapmer motif. In certain such embodiments, the mismatch is at position 1, 2, 3, 4, 5, 6, 7, 8, 9, 10, 11, or 12 from the 5′-end of the oligonucleotide. In certain such embodiments, the mismatch is at position, 2, 3, 4, 5, 6, 7, 8, 9, 10, 11, or 12 from the 3′-end of the oligonucleotide.

The location of a non-complementary nucleobase may be at the 5′ end or 3′ end of the compound. Alternatively, the non-complementary nucleobase or nucleobases may be at an internal position of the compound. When two or more non-complementary nucleobases are present, they may be contiguous (i.e. linked) or non-contiguous. In one embodiment, a non-complementary nucleobase is located in the wing segment of a gapmer oligonucleotide.

In certain embodiments, compounds described herein that are, or are up to 11, 12, 13, 14, 15, 16, 17, 18, 19, or 20 nucleobases in length comprise no more than 4, no more than 3, no more than 2, or no more than 1 non-complementary nucleobase(s) relative to a target nucleic acid, such as a target gene nucleic acid, or specified portion thereof.

In certain embodiments, compounds described herein that are, or are up to 11, 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as a target gene nucleic acid, or specified portion thereof.

In certain embodiments, compounds described herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of a compound. In certain embodiments, the compounds are complementary to at least an 8 nucleobase portion of a target segment. In certain embodiments, the compounds are complementary to at least a 9 nucleobase portion of a target segment. In certain embodiments, the compounds are complementary to at least a 10 nucleobase portion of a target segment. In certain embodiments, the compounds are complementary to at least an 11 nucleobase portion of a target segment. In certain embodiments, the compounds are complementary to at least a 12 nucleobase portion of a target segment. In certain embodiments, the compounds are complementary to at least a 13 nucleobase portion of a target segment. In certain embodiments, the compounds are complementary to at least a 14 nucleobase portion of a target segment. In certain embodiments, the compounds are complementary to at least a 15 nucleobase portion of a target segment. In certain embodiments, the compounds are complementary to at least a 16 nucleobase portion of a target segment. Also contemplated are compounds that are complementary to at least a 9, 10, 11, 12, 13, 14, 15, 16, 17, 18, 19, 20, or more nucleobase portion of a target segment, or a range defined by any two of these values.

### Identity

The compounds provided herein may also have a defined percent identity to a particular nucleotide sequence, SEQ ID NO, or compound represented by a specific Isis number, or portion thereof. In certain embodiments, compounds described herein are antisense compounds or oligomeric compounds. In certain embodiments, compounds described herein are modified oligonucleotides. As used herein, a compound is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the compounds described herein as well as compounds having non-identical bases relative to the compounds provided herein also are contemplated. The non-identical bases may be adjacent to each other or dispersed throughout the compound. Percent identity of a compound is calculated according to the number of bases that have identical base pairing relative to the sequence to which it is being compared.

In certain embodiments, compounds described herein, or portions thereof, are, or are at least, 70%, 75%, 80%, 85%, 90%, 91%, 92%, 93%, 94%, 95%, 96%, 97%, 98%, 99% or 100% identical to one or more of the compounds or SEQ ID NOs, or a portion thereof, disclosed herein. In certain embodiments, compounds described herein are about 70%, 75%, 80%, 85%, 90%, 91%, 92%, 93%, 94%, 95%, 96%, 97%, 98%, or 99% identical, or any percentage between such values, to a particular nucleotide sequence, SEQ ID NO, or compound represented by a specific Isis number, or portion thereof, in which the compounds comprise an oligonucleotide having one or more mismatched nucleobases. In certain such embodiments, the mismatch is at position 1, 2, 3, 4, 5, 6, 7, 8, 9, 10, 11, or 12 from the 5′-end of the oligonucleotide. In certain such embodiments, the mismatch is at position 2, 3, 4, 5, 6, 7, 8, 9, 10, 11, or 12 from the 3′-end of the oligonucleotide.

In certain embodiments, compounds described herein are antisense compounds. In certain embodiments, a portion of the compound is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

In certain embodiments, compounds described herein are oligonucleotides. In certain embodiments, a portion of the oligonucleotide is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

### Certain Modified Compounds

In certain embodiments, compounds described herein comprise or consist of oligonucleotides consisting of linked nucleosides. Oligonucleotides may be unmodified oligonucleotides (RNA or DNA) or may be modified oligonucleotides. Modified oligonucleotides comprise at least one modification relative to unmodified RNA or DNA (i.e., comprise at least one modified nucleoside (comprising a modified sugar moiety and/or a modified nucleobase) and/or at least one modified internucleoside linkage).

A. Modified Nucleosides

Modified nucleosides comprise a modified sugar moiety or a modified nucleobase or both a modifed sugar moiety and a modified nucleobase.

1. Modified Sugar Moieties

In certain embodiments, sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more acyclic substituent, including but not limited to substituents at the 2′, 4′, and/or 5′ positions. In certain embodiments one or more acyclic substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O(CH₂)₂OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for linearlynon-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugars comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.

In certain embodiments, a 2′-substituted nucleoside or 2′-non-bicyclic modified nucleoside comprises a sugar moiety comprising a linear 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside or 2′-non-bicyclic modified nucleoside comprises a sugar moiety comprising a linear 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted nucleoside or 2′-non-bicyclic modified nucleoside comprises a sugar moiety comprising a linear 2′-substituent group selected from: F, OCH₃, and OCH₂CH₂OCH₃.

Nucleosides comprising modified sugar moieties, such as non-bicyclic modified sugar moieties, are referred to by the position(s) of the substitution(s) on the sugar moiety of the nucleoside. For example, nucleosides comprising 2′-substituted or 2-modified sugar moieties are referred to as 2′-substituted nucleosides or 2-modified nucleosides.

Certain modified sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C—(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b}), —C(Rₐ)═N—, —C(═NRₐ)—, —C(═O)—, —C(═S)—, —O—, —Si(Rₐ)₂—, —S(═O)ₓ—, and —N(Rₐ)—;

wherein:

x is 0, 1, or 2;

n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 20017, 129, 8362-8379; Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3, 239-243; Wengel et al., U.S. Pat. No. 7,053,207, Imanishi et al., U.S. Pat. No. 6,268,490, Imanishi et al. U.S. Pat. No. 6,770,748, Imanishi et al., U.S. RE44,779; Wengel et al., U.S. Pat. No. 6,794,499, Wengel et al., U.S. Pat. No. 6,670,461; Wengel et al., U.S. Pat. No. 7,034,133, Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191, Torsten et al., WO 2004/106356, Wengel et al., WO 91999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

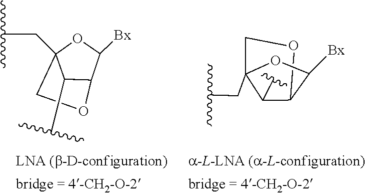

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see e.g., Leumann, C J. Bioorg. & Med. Chem. 2002, 10, 841-854), fluoro HNA:

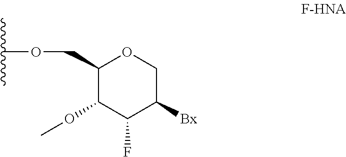

(“F-HNA”, see e.g., Swayze et al., U.S. Pat. No. 8,088,904; Swayze et al., U.S. Pat. No. 8,440,803; Swayze et al., U.S.; and Swayze et al., U.S. Pat. No. 9,005,906, F-HNA can also be referred to as a F-THP or 3′-fluoro tetrahydropyran), and nucleosides comprising additional modified THP compounds having the formula:

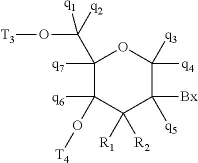

wherein, independently, for each of said modified THP nucleoside: Bx is a nucleobase moiety; T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group; q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

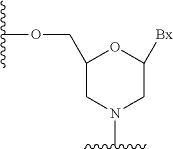

In certain embodiments, morpholinos may be modified, for example by adding or altering various substituent groups from the above morpholino structure. Such sugar surrogates are refered to herein as “modified morpholinos.”

In certain embodiments, sugar surrogates comprise acyclic moieites. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides.

2. Modified Nucleobases

Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications can impart nuclease stability, binding affinity or some other beneficial biological property to compounds described herein.

In certain embodiments, compounds described herein comprise modified oligonucleotides. In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside.

In certain embodiments, modified nucleobases are selected from: 5-substituted pyrimidines, 6-azapyrimi¬dines, alkyl or alkynyl substituted pyrimidines, alkyl substituted purines, and N-2, N-6 and O-6 substituted purines. In certain embodiments, modified nucleobases are selected from: 2-aminopropyladenine, 5-hydroxymethyl cytosine, 5-methylcytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (C≡C—CH3) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403, Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

In certain embodiments, compounds targeted to a target gene nucleic acid comprise one or more modified nucleobases. In certain embodiments, the modified nucleobase is 5-methylcytosine. In certain embodiments, each cytosine is a 5-methylcytosine.

### Modified Internucleoside Linkages

The naturally occurring internucleoside linkage of RNA and DNA is a 3′ to 5′ phosphodiester linkage. In certain embodiments, compounds described herein having one or more modified, i.e. non-naturally occurring, internucleoside linkages are often selected over compounds having naturally occurring internucleoside linkages because of desirable properties such as, for example, enhanced cellular uptake, enhanced affinity for target nucleic acids, and increased stability in the presence of nucleases.

In certain embodiments, compounds targeted to a target gene nucleic acid comprise one or more modified internucleoside linkages. In certain embodiments, the modified internucleoside linkages are phosphorothioate linkages. In certain embodiments, each internucleoside linkage of the compound is a phosphorothioate internucleoside linkage.

In certain embodiments, compounds described herein comprise oligonucleotides. Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linking groups include but are not limited to methylenemethylimino (—CH2-N(CH3)-O—CH2-), thiodiester, thionocarbamate (—O—C(═O)(NH)—S—); siloxane (—O—SiH2-O—); and N,N′-dimethylhydrazine (—CH2-N(CH3)-N(CH3)-). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Representative chiral internucleoside linkages include but are not limited to alkylphosphonates and phosphorothioates. Methods of preparation of phosphorous-containing and non-phosphorous-containing internucleoside linkages are well known to those skilled in the art.

Neutral internucleoside linkages include, without limitation, phosphotriesters, methylphosphonates, MMI (3′-CH2-N(CH3)-O-5′), amide-3 (3′-CH2-C(═O)—N(H)-5′), amide-4 (3′-CH2-N(H)—C(═O)-5′), formacetal (3′-O—CH2-O-5′), methoxypropyl, and thioformacetal (3′-S—CH2-O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH2 component parts.

In certain embodiments, oligonucleotides comprise modified internucleoside linkages arranged along the oligonucleotide or region thereof in a defined pattern or modified internucleoside linkage motif. In certain embodiments, internucleoside linkages are arranged in a gapped motif. In such embodiments, the internucleoside linkages in each of two wing regions are different from the internucleoside linkages in the gap region. In certain embodiments the internucleoside linkages in the wings are phosphodiester and the internucleoside linkages in the gap are phosphorothioate. The nucleoside motif is independently selected, so such oligonucleotides having a gapped internucleoside linkage motif may or may not have a gapped nucleoside motif and if it does have a gapped nucleoside motif, the wing and gap lengths may or may not be the same.

In certain embodiments, oligonucleotides comprise a region having an alternating internucleoside linkage motif. In certain embodiments, oligonucleotides of the present invention comprise a region of uniformly modified internucleoside linkages. In certain such embodiments, the oligonucleotide comprises a region that is uniformly linked by phosphorothioate internucleoside linkages. In certain embodiments, the oligonucleotide is uniformly linked by phosphorothioate. In certain embodiments, each internucleoside linkage of the oligonucleotide is selected from phosphodiester and phosphorothioate. In certain embodiments, each internucleoside linkage of the oligonucleotide is selected from phosphodiester and phosphorothioate and at least one internucleoside linkage is phosphorothioate.

In certain embodiments, the oligonucleotide comprises at least 6 phosphorothioate internucleoside linkages. In certain embodiments, the oligonucleotide comprises at least 8 phosphorothioate internucleoside linkages. In certain embodiments, the oligonucleotide comprises at least 10 phosphoro-thioate internucleoside linkages. In certain embodiments, the oligonucleotide comprises at least one block of at least 6 consecutive phosphorothioate internucleoside linkages. In certain embodiments, the oligonucleotide comprises at least one block of at least 8 consecutive phosphorothioate internucleoside linkages. In certain embodiments, the oligonucleotide comprises at least one block of at least 10 consecutive phosphorothioate internucleoside linkages. In certain embodiments, the oligonucleotide comprises at least block of at least one 12 consecutive phosphorothioate internucleoside linkages. In certain such embodiments, at least one such block is located at the 3′ end of the oligonucleotide. In certain such embodiments, at least one such block is located within 3 nucleosides of the 3′ end of the oligonucleotide.

In certain embodiments, oligonucleotides comprise one or more methylphosponate linkages. In certain embodiments, oligonucleotides having a gapmer nucleoside motif comprise a linkage motif comprising all phosphorothioate linkages except for one or two methylphosponate linkages. In certain embodiments, one methylphosponate linkage is in the central gap of an oligonucleotide having a gapmer nucleoside motif.

In certain embodiments, it is desirable to arrange the number of phosphorothioate internucleoside linkages and phosphodiester internucleoside linkages to maintain nuclease resistance. In certain embodiments, it is desirable to arrange the number and position of phosphorothioate internucleoside linkages and the number and position of phosphodiester internucleoside linkages to maintain nuclease resistance. In certain embodiments, the number of phosphorothioate internucleoside linkages may be decreased and the number of phosphodiester internucleoside linkages may be increased. In certain embodiments, the number of phosphorothioate internucleoside linkages may be decreased and the number of phosphodiester internucleoside linkages may be increased while still maintaining nuclease resistance. In certain embodiments it is desirable to decrease the number of phosphorothioate internucleoside linkages while retaining nuclease resistance. In certain embodiments it is desirable to increase the number of phosphodiester internucleoside linkages while retaining nuclease resistance.

B. Certain Motifs

In certain embodiments, compounds described herein comprise oligonucleotides. Oligonucleotides can have a motif, e.g. a pattern of unmodified and/or modified sugar moieties, nucleobases, and/or internucleoside linkages. In certain embodiments, modified oligonucleotides comprise one or more modified nucleoside comprising a modified sugar. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

1. Certain Sugar Motifs

In certain embodiments, compounds described herein comprise oligonucleotides. In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, modified oligonucleotides comprise or consist of a region having a gapmer motif, which comprises two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-5 nucleosides. In certain embodiments, the wings of a gapmer comprise 2-5 nucleosides. In certain embodiments, the wings of a gapmer comprise 3-5 nucleosides. In certain embodiments, the nucleosides of a gapmer are all modified nucleosides.

In certain embodiments, the gap of a gapmer comprises 7-12 nucleosides. In certain embodiments, the gap of a gapmer comprises 7-10 nucleosides. In certain embodiments, the gap of a gapmer comprises 8-10 nucleosides. In certain embodiments, the gap of a gapmer comprises 10 nucleosides. In certain embodiment, each nucleoside of the gap of a gapmer is an unmodified 2′-deoxy nucleoside.

In certain embodiments, the gapmer is a deoxy gapmer. In such embodiments, the nucleosides on the gap side of each wing/gap junction are unmodified 2′-deoxy nucleosides and the nucleosides on the wing sides of each wing/gap junction are modified nucleosides. In certain such embodiments, each nucleoside of the gap is an unmodified 2′-deoxy nucleoside. In certain such embodiments, each nucleoside of each wing is a modified nucleoside.

In certain embodiments, a modified oligonucleotide has a fully modified sugar motif wherein each nucleoside of the modified oligonucleotide comprises a modified sugar moiety. In certain embodiments, modified oligonucleotides comprise or consist of a region having a fully modified sugar motif wherein each nucleoside of the region comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified comprises the same 2′-modification.

2. Certain Nucleobase Motifs

In certain embodiments, compounds described herein comprise oligonucleotides. In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl moiety. In certain embodiments, the modified nucleobase is selected from: a 2-thiopyrimidine and a 5-propynepyrimidine.

3. Certain Internucleoside Linkage Motifs

In certain embodiments, compounds described herein comprise oligonucleotides. In certain embodiments, oligonucleotides comprise modified and/or unmodified internucleoside linkages arranged along the oligonucleotide or region thereof in a defined pattern or motif. In certain embodiments, essentially each internucleoside linking group is a phosphate internucleoside linkage (P═O). In certain embodiments, each internucleoside linking group of a modified oligonucleotide is a phosphorothioate (P═S). In certain embodiments, each internucleoside linking group of a modified oligonucleotide is independently selected from a phosphorothioate and phosphate internucleoside linkage. In certain embodiments, the sugar motif of a modified oligonucleotide is a gapmer and the internucleoside linkages within the gap are all modified. In certain such embodiments, some or all of the internucleoside linkages in the wings are unmodified phosphate linkages. In certain embodiments, the terminal internucleoside linkages are modified.

C. Certain Modified Oligonucleotides

In certain embodiments, compounds described herein comprise modified oligonucleotides. In certain embodiments, the above modifications (sugar, nucleobase, internucleoside linkage) are incorporated into a modified oligonucleotide. In certain embodiments, modified oligonucleotides are characterized by their modification, motifs, and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such gapmer oligonucleotides may comprise one or more modified nucleobase independent of the gapmer pattern of the sugar modifications. Furthermore, in certain instances, an oligonucleotide is described by an overall length or range and by lengths or length ranges of two or more regions (e.g., a regions of nucleosides having specified sugar modifications), in such circumstances it may be possible to select numbers for each range that result in an oligonucleotide having an overall length falling outside the specified range. In such circumstances, both elements must be satisfied. For example, in certain embodiments, a modified oligonucleotide consists of 15-20 linked nucleosides and has a sugar motif consisting of three regions, A, B, and C, wherein region A consists of 2-6 linked nucleosides having a specified sugar motif, region B consists of 6-10 linked nucleosides having a specified sugar motif, and region C consists of 2-6 linked nucleosides having a specified sugar motif. Such embodiments do not include modified oligonucleotides where A and C each consist of 6 linked nucleosides and B consists of 10 linked nucleosides (even though those numbers of nucleosides are permitted within the requirements for A, B, and C) because the overall length of such oligonucleotide is 22, which exceeds the upper limit of the overall length of the modified oligonucleotide (20). Herein, if a description of an oligonucleotide is silent with respect to one or more parameter, such parameter is not limited. Thus, a modified oligonucleotide described only as having a gapmer sugar motif without further description may have any length, internucleoside linkage motif, and nucleobase motif Unless otherwise indicated, all modifications are independent of nucleobase sequence.

### Compositions and Methods for Formulating Pharmaceutical Compositions

Compounds described herein may be admixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

In certain embodiments, the present invention provides pharmaceutical compositions comprising one or more compounds or a salt thereof. In certain embodiments, the compounds are antisense compounds or oligomeric compounds. In certain embodiments, the compounds comprise or consist of a modified oligonucleotide. In certain such embodiments, the pharmaceutical composition comprises a suitable pharmaceutically acceptable diluent or carrier. In certain embodiments, a pharmaceutical composition comprises a sterile saline solution and one or more compound. In certain embodiments, such pharmaceutical composition consists of a sterile saline solution and one or more compound. In certain embodiments, the sterile saline is pharmaceutical grade saline. In certain embodiments, a pharmaceutical composition comprises one or more compound and sterile water. In certain embodiments, a pharmaceutical composition consists of one compound and sterile water. In certain embodiments, the sterile water is pharmaceutical grade water. In certain embodiments, a pharmaceutical composition comprises one or more compound and phosphate-buffered saline (PBS). In certain embodiments, a pharmaceutical composition consists of one or more compound and sterile PBS. In certain embodiments, the sterile PBS is pharmaceutical grade PBS. Compositions and methods for the formulation of pharmaceutical compositions are dependent upon a number of criteria, including, but not limited to, route of administration, extent of disease, or dose to be administered.

A compound described herein targeted to a target gene nucleic acid can be utilized in pharmaceutical compositions by combining the compound with a suitable pharmaceutically acceptable diluent or carrier. In certain embodiments, a pharmaceutically acceptable diluent is water, such as sterile water suitable for injection. Accordingly, in one embodiment, employed in the methods described herein is a pharmaceutical composition comprising a compound targeted to a target gene nucleic acid and a pharmaceutically acceptable diluent. In certain embodiments, the pharmaceutically acceptable diluent is water. In certain embodiments, the compound comprises or consists of a modified oligonucleotide provided herein.

Pharmaceutical compositions comprising compounds provided herein encompass any pharmaceutically acceptable salts, esters, or salts of such esters, or any other oligonucleotide which, upon administration to an individual, including a human, is capable of providing (directly or indirectly) the biologically active metabolite or residue thereof. In certain embodiments, the compounds are antisense compounds or oligomeric compounds. In certain embodiments, the compound comprises or consists of a modified oligonucleotide. Accordingly, for example, the disclosure is also drawn to pharmaceutically acceptable salts of compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of a compound which are cleaved by endogenous nucleases within the body, to form the active compound.

In certain embodiments, the compounds or compositions further comprise a pharmaceutically acceptable carrier or diluent.

### EXAMPLES

### Non-Limiting Disclosure and Incorporation by Reference

While certain compounds, compositions and methods described herein have been described with specificity in accordance with certain embodiments, the following examples serve only to illustrate the compounds described herein and are not intended to limit the same. Each of the references recited in the present application is incorporated herein by reference in its entirety.

### Example 1: Generation of HEK Cells Ectopically Expressing ASGPR

Human embryonic kidney HEK 293 cells stably expressing active ASGPR were generated to directly compare ASO uptake and potencies conferred by ASGPR. Stably transfected HEK cell lines expressing the ASGPR1 and ASGPR2 individually or concurrently were created to test the relative importance of the two ASGPR sub-units in ASO uptake. The ASGPR is composed of both a major (ASGPR1) and minor subunit (ASGPR2). ASGPR1 has been shown to be efficiently targeted to the plasma membrane and to undergo constitutive endocytosis and recycling.

ASGPR1 was observed to display a distribution split between the plasma membrane and endomembrane compartments representing ASGPR1 in the endocytic pathway. Furthermore, western blot of cells stably expressing ASGPR1 produced a single band migrating at approximately 45 kDa, consistent with the mature glycosylated, post-golgi species. ASGPR2 lacks the ER export signal present in ASGPR1 and has been reported to be largely retained in the ER and rapidly degraded. No evidence of ASGPR2 delivery to the plasma membrane was detected; rather dim endomembrane staining indicative of biosynthetic localization was observed. Western blot of these cells revealed a predominate ASGPR2 species migrating at ˜40 kDa, representing immature ASGPR2, with minor diffuse higher molecular weight bands likely to be ASGPR2 that escaped the ER and underwent mature glycosylation. ASGPR2 cells infected with ASGPR1 virus revealed an overall higher ASGPR2 immunofluorescence and the appearance of ASGPR2 at the plasma membrane. Co-expression of ASGPR1 resulted in the appearance of dominant higher migrating ASGPR2 immunoreactive bands on western blots.

Uptake of the phosphodiester ASO GalNAc3 conjugate was evaluated and robust uptake was observed only in ASGRP1-expressing clones. When uptake of phosphorothioate containing ASOs was measured in the ASGR1-expressing cells a pronounced increase in uptake of the GalNAc-conjugate relative to the parent ASO that was eliminated by free GalNAc was observed, whereas uptake of the parent ASO was slightly increased by excess free GalNAc. When this line was tested in free uptake activity assays the GalNAc conjugate was 18-fold more potent than the parent ASO and this improvement was likewise eliminated by excess free GalNAc. When activity of the GalNAc-ASO conjugate was tested in the ASGR1/ASGR2 co-expressing cell line we found no discernable difference from the ASGR1 only expressing line.

### Example 2: Uptake of GalNAc Conjugated ASOs in ASGPR1 Inducible HEK Cells

An ASGPR1 inducible cell line, TREX-ASGPR1 was created. In the absence of doxycycline, uptake of the parent and GalNAc-conjugate ASO were very similar. Following 24 hours in the presence of doxycycline, however, there was 2-3 fold increased uptake of the GalNAc ASO conjugate, similar to the non-inducible stable cell line.

Interestingly, ASGPR1 induction also produced a small but consistent increase in uptake of the unconjugated parent, suggesting ASGPR1 may promote uptake of phosphorothioate containing ASOs. When TREX ASGPR1 cells were tested in activity assays in the absence of doxycycline induction, the ASO parent and conjugate ASO yielded IC50's of approximately one and two micromolar, respectively, similar to untransfected HEK 293 cells. ASGPR1 induction produced a dramatic decrease in the IC50 of the GalNAc-conjugated ASO that was more than 100 fold lower than in the non-induced cells. A 2-3 fold decrease of the parent ASO under ASGPR induction was observed.

### Example 3: Gene Therapy Delivery of ASGPR and Administration of a GalNAc-Conjugated ASO In Vivo

Adeno-associated virus 9 (AAV9) vector carrying human ASGPR1 is prepared by standard AAV helper-free methods. Briefly, the human ASGPR gene is cloned into to ITR/MCS of an AAV9-ITR-containing plasmid driven by a CMV promotor. The resulting recombinant plasmid is transiently co-transfected into HEK293 cells along with a plasmid encoding Rep-Cap and a plasmid (pHelper) encoding adenovirus helper genes under standard transfection conditions. Briefly, HEK293T cells are plated at 3×10⁶ cells per 100-mm tissue culture plate in 10 mL DMEM 48 hours prior to transfection. The HEK293T cells are transfected by calcium-phosphate. Recombinant AAV9 viral particles are produced by the infected HEK293 cells. Viral stock is prepared from the growth medium and cell suspension three freeze-thaw cycles and centrifugation at 10,000×g. The supernatant is saved as virus stock. Viral titer is determined by PCR or slot blot. Additional details are described in Shin J H et al., 2012 Methods Mol Biol 798:267-284, which is incorporated by reference in its entirety herein.

4.32E+10 viral particles carrying ASGPR1 (AAV-ASGPR1) were administered to 10 week wild-type C57Bl/6 mice by injection into the striatum at the coordinates: +0.5 mm AP, ±2 mm L, −3 mm DV. Injection was performed with Hamilton syringe (5 ul-model 75): 7634-01 with Hamilton custom stainless steel needles (33 Gauge; O.D×I.D. (mm) −0.21×0.11): 7803-05, 33GA RN 6PK 0.375” PT30°.

2 weeks after the mice were injected with the AAV-ASGPR1, the mice were administered an intracerebroventricular (ICV) bolus injection of approximately 90 μg of unconjugated MALAT1 antisense oligonucleotide ISIS 626112 (GCCAGGCTGGTTATGACTCA; SEQ ID NO: 1) or approximately 109 μg of GalNAc conjugated MALAT1 antisense oligonucleotide ION 1010428 having the same sequence. Both ISIS 626112 and ION 1010428 are 5-10-5 MOE gapmers with mixed backbones.

The mice were sacrificed two weeks after ICV injection of the antisense oligonucleotide (ASO) and brain tissue sections were obtained for measuring expression levels of ASGPR1 and ASO by immunohistochemistry (IHC) and levels of MALAT1 RNA by in situ hybridization.

Intra-striatal injection of AAV-ASGPR1 led to broad ASGPR1 expression in the central nervous system (CNS). IHC staining showed some correlation between ASGPR1 expression and GalNAc conjugated ASO accumulation in certain regions of the brain and spinal cord. GalNAc conjugated ASO reduced MALAT1 levels in certain regions of the striatum and spinal cord having relatively higher levels of ASGPR1 expression compared to adjacent areas having relatively lower levels of ASGPR1 expression.

In a similar experiment, 2 weeks after mice were injected with the AAV-ASGPR1, the mice were administered an intracerebroventricular (ICV) bolus injection of approximately 300 μg of unconjugated HDAC2 antisense oligonucleotide ISIS 677469 (CTCACTTTTCGAGGTTCCTA; SEQ ID NO: 2) or approximately 364 μg of GalNAc conjugated HDAC2 antisense oligonucleotide ION 1010429 having the same sequence. Both ISIS 677469 and ION 1010429 are 5-10-5 MOE gapmers with mixed backbones. IHC staining showed some correlation between ASGPR1 expression and GalNAc conjugated ASO accumulation broadly in certain regions of the brain. HDAC2 expression level was not assessed.

1. A method comprising:
contacting a non-native cell with a cell surface receptor upregulating agent, thereby generating a non-native cell ectopically expressing the cell surface receptor; and
contacting the non-native cell ectopically expressing the cell surface receptor with a compound comprising a modified oligonucleotide and a conjugate group, wherein the conjugate group binds to the cell surface receptor.

2. A method comprising contacting a non-native cell ectopically expressing a cell surface receptor with a compound comprising a modified oligonucleotide and a conjugate group, wherein the conjugate group binds to the cell surface receptor.

3. The method of claim 1 wherein the non-native cell is a non-liver cell.

4-6. (canceled)

7. The method of claim 3, wherein the non-native cell is an eye, muscle, heart, skin, kidney, lung, pancreas, intestinal, fat, spleen, bone, testes, ovary, pituitary, immune, or bladder cell, or a cell in the central nervous system (CNS).

8. The method of claim 1, wherein the cell surface receptor is a liver cell receptor.

9. The method of claim 8, wherein the cell surface receptor is asialoglycoprotein receptor (ASGPR).

10. The method of claim 9, wherein the ASGPR is the ASGPR subunit 1 (ASGPR1).

11. The method of claim 9, wherein the conjugate group comprises N-acetyl galactosamine (GalNAc).

12. The method of claim 11 wherein the conjugate group comprises:

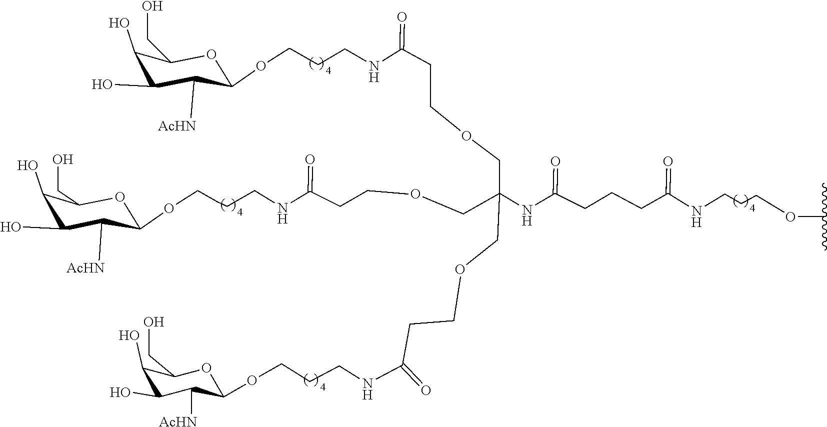

13. The method of claim 1, wherein the cell surface receptor upregulating agent comprises a vector and a nucleic acid encoding the cell surface receptor; a modified oligonucleotide complementary to a target site with a translation suppression element region of a RNA transcript encoding a cell surface receptor; a RNA transcript encoding a cell surface receptor; or a CRISPR system homology directed repair insertion cassette comprising a nucleic acid encoding a cell surface receptor.

14-17. (canceled)

18. The method of claim 13, wherein the vector comprises a virus.

19-24. (canceled)

25. The method of claim 1, wherein the compound is single-stranded.

26. (canceled)

27. The method of claim 1, wherein the modified oligonucleotide is 12 to 30 linked nucleosides in length.

28. The method of claim 27, wherein the modified oligonucleotide comprises at least one modified internucleoside linkage, at least one modified sugar moiety, or at least one modified nucleobase.

29. The method of claim 28, wherein at least one modified sugar comprises a 2′-O-methyoxyethyl or a bicyclic sugar selected from the group consisting of: 4′-(CH₂)—O-2′ (LNA); 4′-(CH₂)₂—O-2′ (ENA); and 4′-CH(CH₃)—O-2′ (cEt).

30-31. (canceled)

32. The method of claim 1, wherein the modified oligonucleotide comprises:
a gap segment consisting of linked deoxynucleosides;
a 5′ wing segment consisting of linked nucleosides;
a 3′ wing segment consisting linked nucleosides;
wherein the gap segment is positioned immediately adjacent to and between the 5′ wing segment and the 3′ wing segment and wherein each nucleoside of each wing segment comprises a modified sugar.

33-34. (canceled)

35. The method of claim 1, wherein:
said contacting a non-native cell with a cell surface receptor upregulating agent, comprises administering to a subject the cell surface receptor upregulating agent, thereby generating the non-native cell ectopically expressing the cell surface receptor in the subject; and
said contacting the non-native cell ectopically expressing the cell surface receptor with a compound comprises administering to the subject the compound comprising a modified oligonucleotide and a conjugate group, wherein the conjugate group binds to the cell surface receptor, thereby delivering the compound to the non-native cell ectopically expressing the cell surface receptor in the subject.

36. The method of claim 2, wherein said contacting a non-native cell ectopically expressing a cell surface receptor with a compound comprising a modified oligonucleotide and a conjugate group, wherein the conjugate group binds to the cell surface receptor comprises administering to a subject having a cell ectopically expressing a cell surface receptor the compound comprising a modified oligonucleotide and a conjugate group, wherein the conjugate group binds to the cell surface receptor.

37-63. (canceled)

64. The method of claim 35, wherein the cell surface upregulating agent or compound are administered to the subject by direct injection to the tissue comprising the non-native cell.

65. The method of claim 64, wherein the tissue is the brain or eye.


SEQUENCE LISTING (2 sequences)

SEQ ID NO: 1 - DNA, 20 bp, synthetic construct
  source 1..20: mol_type = other DNA
gccaggctggttatgactca

SEQ ID NO: 2 - DNA, 20 bp, synthetic construct
  source 1..20: mol_type = other DNA
ctcacttttcgaggttccta